SEQUENCE LISTING (86 sequences)

SEQ ID NO: 1 - DNA, 22 bp, synthetic construct
  misc_feature 1..22: note = Synthetic
  source 1..22: mol_type = other DNA
agcaaagaggtcctgatggaga

SEQ ID NO: 2 - DNA, 20 bp, synthetic construct
  misc_feature 1..20: note = Synthetic
  source 1..20: mol_type = other DNA
gcagggcccatcacacacat

SEQ ID NO: 3 - DNA, 28 bp, synthetic construct
  misc_feature 1..28: note = Synthetic
  source 1..28: mol_type = other DNA
aagttattaagtttatggtagtgtgtta

SEQ ID NO: 4 - DNA, 24 bp, synthetic construct
  misc_feature 1..24: note = Synthetic
  source 1..24: mol_type = other DNA
atccacaaaattaaaaacaacact

SEQ ID NO: 5 - DNA, 22 bp, synthetic construct
  misc_feature 1..22: note = Synthetic
  source 1..22: mol_type = other DNA
gttgttgggtgaggtagtttta

SEQ ID NO: 6 - DNA, 23 bp, synthetic construct
  misc_feature 1..23: note = Synthetic
  source 1..23: mol_type = other DNA
ttcactaaatctttccaaatcac

SEQ ID NO: 7 - DNA, 27 bp, synthetic construct
  misc_feature 1..27: note = Synthetic
  source 1..27: mol_type = other DNA
ttagattttaggatttagatatttatt

SEQ ID NO: 8 - DNA, 29 bp, synthetic construct
  misc_feature 1..29: note = Synthetic
  source 1..29: mol_type = other DNA
tcttaactatacacaaaaaactactatac

SEQ ID NO: 9 - DNA, 23 bp, synthetic construct
  misc_feature 1..23: note = Synthetic
  source 1..23: mol_type = other DNA
ttatgatagggaaaaataaggtg

SEQ ID NO: 10 - DNA, 24 bp, synthetic construct
  misc_feature 1..24: note = Synthetic
  source 1..24: mol_type = other DNA
caaaacccaatctaattacttaac

SEQ ID NO: 11 - DNA, 25 bp, synthetic construct
  misc_feature 1..25: note = Synthetic
  source 1..25: mol_type = other DNA
ttttttaaagtgttagaattatagg

SEQ ID NO: 12 - DNA, 23 bp, synthetic construct
  misc_feature 1..23: note = Synthetic
  source 1..23: mol_type = other DNA
atttaacttaaaattcactttcc

SEQ ID NO: 13 - DNA, 26 bp, synthetic construct
  misc_feature 1..26: note = Synthetic
  source 1..26: mol_type = other DNA
ttaaataaaaagttaaagttaaaatg

SEQ ID NO: 14 - DNA, 25 bp, synthetic construct
  misc_feature 1..25: note = Synthetic
  source 1..25: mol_type = other DNA
taattcctaaactaataacatccac

SEQ ID NO: 15 - DNA, 20 bp, synthetic construct
  misc_feature 1..20: note = Synthetic
  source 1..20: mol_type = other DNA
ggttgtttgtttgtgtttaa

SEQ ID NO: 16 - DNA, 22 bp, synthetic construct
  misc_feature 1..22: note = Synthetic
  source 1..22: mol_type = other DNA
acacaaacaacaatacttaaaa

SEQ ID NO: 17 - DNA, 22 bp, synthetic construct
  misc_feature 1..22: note = Synthetic
  source 1..22: mol_type = other DNA
tgttttgtttgggagttagttg

SEQ ID NO: 18 - DNA, 25 bp, synthetic construct
  misc_feature 1..25: note = Synthetic
  source 1..25: mol_type = other DNA
aacaaatactaaacttcttccaaaa

SEQ ID NO: 19 - DNA, 27 bp, synthetic construct
  misc_feature 1..27: note = Synthetic
  source 1..27: mol_type = other DNA
attttatagtgtgttatttagaagaga

SEQ ID NO: 20 - DNA, 28 bp, synthetic construct
  misc_feature 1..28: note = Synthetic
  source 1..28: mol_type = other DNA
atataaaaaataaacaacttaaactatc

SEQ ID NO: 21 - DNA, 27 bp, synthetic construct
  misc_feature 1..27: note = Synthetic
  source 1..27: mol_type = other DNA
gttttataggtttagatattttagttt

SEQ ID NO: 22 - DNA, 24 bp, synthetic construct
  misc_feature 1..24: note = Synthetic
  source 1..24: mol_type = other DNA
caaccaaaactaaaaataaatacc

SEQ ID NO: 23 - DNA, 29 bp, synthetic construct
  misc_feature 1..29: note = Synthetic
  source 1..29: mol_type = other DNA
ttttatgtatagattaatagtaaagtttt

SEQ ID NO: 24 - DNA, 25 bp, synthetic construct
  misc_feature 1..25: note = Synthetic
  source 1..25: mol_type = other DNA
aaaccaacatttctctataactact

SEQ ID NO: 25 - DNA, 21 bp, synthetic construct
  misc_feature 1..21: note = Synthetic
  source 1..21: mol_type = other DNA
attttttttggttggattgtt

SEQ ID NO: 26 - DNA, 20 bp, synthetic construct
  misc_feature 1..20: note = Synthetic
  source 1..20: mol_type = other DNA
tcacaaacacacaaacccaa

SEQ ID NO: 27 - DNA, 22 bp, synthetic construct
  misc_feature 1..22: note = Synthetic
  source 1..22: mol_type = other DNA
ttttaaagaagtttttatgggt

SEQ ID NO: 28 - DNA, 23 bp, synthetic construct
  misc_feature 1..23: note = Synthetic
  source 1..23: mol_type = other DNA
ataaaccaaacaacactacacat

SEQ ID NO: 29 - DNA, 24 bp, synthetic construct
  misc_feature 1..24: note = Synthetic
  source 1..24: mol_type = other DNA
aggttgtttttttattttttagat

SEQ ID NO: 30 - DNA, 22 bp, synthetic construct
  misc_feature 1..22: note = Synthetic
  source 1..22: mol_type = other DNA
tttccctccctttaataactat

SEQ ID NO: 31 - DNA, 25 bp, synthetic construct
  misc_feature 1..25: note = Synthetic
  source 1..25: mol_type = other DNA
ttagtttttttagtatgattttgag

SEQ ID NO: 32 - DNA, 23 bp, synthetic construct
  misc_feature 1..23: note = Synthetic
  source 1..23: mol_type = other DNA
caccacaaaaatcacaatactat

SEQ ID NO: 33 - DNA, 25 bp, synthetic construct
  misc_feature 1..25: note = Synthetic
  source 1..25: mol_type = other DNA
gttaagaaattaataggaaaaagaa

SEQ ID NO: 34 - DNA, 20 bp, synthetic construct
  misc_feature 1..20: note = Synthetic
  source 1..20: mol_type = other DNA
aaaaccccatattacttccc

SEQ ID NO: 35 - DNA, 23 bp, synthetic construct
  misc_feature 1..23: note = Synthetic
  source 1..23: mol_type = other DNA
ggggtattttttattattttatt

SEQ ID NO: 36 - DNA, 22 bp, synthetic construct
  misc_feature 1..22: note = Synthetic
  source 1..22: mol_type = other DNA
acacacaacttcaaaaacttca

SEQ ID NO: 37 - DNA, 25 bp, synthetic construct
  misc_feature 1..25: note = Synthetic
  source 1..25: mol_type = other DNA
ggagttgttgtagtagttttttaga

SEQ ID NO: 38 - DNA, 22 bp, synthetic construct
  misc_feature 1..22: note = Synthetic
  source 1..22: mol_type = other DNA
aaattccacaatactcccacta

SEQ ID NO: 39 - DNA, 24 bp, synthetic construct
  misc_feature 1..24: note = Synthetic
  source 1..24: mol_type = other DNA
tttgagagttgtttataatagggt

SEQ ID NO: 40 - DNA, 20 bp, synthetic construct
  misc_feature 1..20: note = Synthetic
  source 1..20: mol_type = other DNA
cctcccttctccacaaacta

SEQ ID NO: 41 - DNA, 22 bp, synthetic construct
  misc_feature 1..22: note = Synthetic
  source 1..22: mol_type = other DNA
tgaaggaaatgagagtaaaggt

SEQ ID NO: 42 - DNA, 20 bp, synthetic construct
  misc_feature 1..20: note = Synthetic
  source 1..20: mol_type = other DNA
cccttctccctaaaaaaaac

SEQ ID NO: 43 - DNA, 25 bp, synthetic construct
  misc_feature 1..25: note = Synthetic
  source 1..25: mol_type = other DNA
tttgttaggttaagtaatttgtaaa

SEQ ID NO: 44 - DNA, 25 bp, synthetic construct
  misc_feature 1..25: note = Synthetic
  source 1..25: mol_type = other DNA
catctcctacttaaataacttcaat

SEQ ID NO: 45 - DNA, 21 bp, synthetic construct
  misc_feature 1..21: note = Synthetic
  source 1..21: mol_type = other DNA
tgtaggtattttagattgggg

SEQ ID NO: 46 - DNA, 23 bp, synthetic construct
  misc_feature 1..23: note = Synthetic
  source 1..23: mol_type = other DNA
aattatccaactcctcactctta

SEQ ID NO: 47 - DNA, 25 bp, synthetic construct
  misc_feature 1..25: note = Synthetic
  source 1..25: mol_type = other DNA
ttttaagaagtttttgtgttattat

SEQ ID NO: 48 - DNA, 24 bp, synthetic construct
  misc_feature 1..24: note = Synthetic
  source 1..24: mol_type = other DNA
tctaaaaatacctaaatacaaacc

SEQ ID NO: 49 - DNA, 22 bp, synthetic construct
  misc_feature 1..22: note = Synthetic
  source 1..22: mol_type = other DNA
gtttgttttgagatgtgagaat

SEQ ID NO: 50 - DNA, 23 bp, synthetic construct
  misc_feature 1..23: note = Synthetic
  source 1..23: mol_type = other DNA
ataacatccttacaaactcacaa

SEQ ID NO: 51 - DNA, 23 bp, synthetic construct
  misc_feature 1..23: note = Synthetic
  source 1..23: mol_type = other DNA
ggtgttataggtagggtagagaa

SEQ ID NO: 52 - DNA, 22 bp, synthetic construct
  misc_feature 1..22: note = Synthetic
  source 1..22: mol_type = other DNA
ccaacatttatcattttcttca

SEQ ID NO: 53 - DNA, 21 bp, synthetic construct
  misc_feature 1..21: note = Synthetic
  source 1..21: mol_type = other DNA
agtatttttattgggttggat

SEQ ID NO: 54 - DNA, 24 bp, synthetic construct
  misc_feature 1..24: note = Synthetic
  source 1..24: mol_type = other DNA
cctactacctcaaattaactaaaa

SEQ ID NO: 55 - DNA, 22 bp, synthetic construct
  misc_feature 1..22: note = Synthetic
  source 1..22: mol_type = other DNA
tgggttttgttgttatagtttt

SEQ ID NO: 56 - DNA, 23 bp, synthetic construct
  misc_feature 1..23: note = Synthetic
  source 1..23: mol_type = other DNA
atatctaccctcttctcttcctc

SEQ ID NO: 57 - DNA, 22 bp, synthetic construct
  misc_feature 1..22: note = Synthetic
  source 1..22: mol_type = other DNA
ttaggtttggattttaattttg

SEQ ID NO: 58 - DNA, 21 bp, synthetic construct
  misc_feature 1..21: note = Synthetic
  source 1..21: mol_type = other DNA
ccctaacccttatctactcca

SEQ ID NO: 59 - DNA, 96 bp, synthetic construct
  misc_feature 1..96: note = Synthetic
  source 1..96: mol_type = other DNA
aagctactaagtttatggcagtgtgttacgataacagtaggaaacggacacggatatcttagtattatcgaaagtgttgtttttaaccttgtggac

SEQ ID NO: 60 - DNA, 110 bp, synthetic construct
  misc_feature 1..110: note = Synthetic
  source 1..110: mol_type = other DNA
gctgctgggtgaggcagctttacgaggccgaagtcctccatgtcaggagcacttggcctcggactgtcttctgaggcctcggccatcgtgacctggaaagacccagtgaa

SEQ ID NO: 61 - DNA, 119 bp, synthetic construct
  misc_feature 1..119: note = Synthetic
  source 1..119: mol_type = other DNA
ccagacctcaggacccagacacccattcgcaggtaaactcccaagcggagggggcacctgtgtccaccgaaggaccaggtgcaagaacacgcacagcagcttcctgtgcacagccaaga

SEQ ID NO: 62 - DNA, 133 bp, synthetic construct
  misc_feature 1..133: note = Synthetic
  source 1..133: mol_type = other DNA
ctatgacagggaaaaacaaggtgggaatgcggaaggtgacagcaggaatccgcagttctgtgtgggatatgttaactttgagatgcgtatcagctagctatctgagtacgtcaagcaaccagactgggttttg

SEQ ID NO: 63 - DNA, 68 bp, synthetic construct
  misc_feature 1..68: note = Synthetic
  source 1..68: mol_type = other DNA
cctctcaaagtgctagaattacaggcgtgacccaccgcacccggcggaaagtgaactttaagtcaaac

SEQ ID NO: 64 - DNA, 117 bp, synthetic construct
  misc_feature 1..117: note = Synthetic
  source 1..117: mol_type = other DNA
ctaaacaaaaagtcaaagttaaaatgcgggctccaattcaggaggtccgcgtggggccaagattctgcatttctaaggagcttgcagttgacgtggatgctaccagttcaggaatca

SEQ ID NO: 65 - DNA, 71 bp, synthetic construct
  misc_feature 1..71: note = Synthetic
  source 1..71: mol_type = other DNA
ggttgcctgtctgtgtttaaagtccaaatcgtatcgggttccgttcgtcttccaagcattgttgcttgtgt

SEQ ID NO: 66 - DNA, 87 bp, synthetic construct
  misc_feature 1..87: note = Synthetic
  source 1..87: mol_type = other DNA
tgctctgcctgggagccagctgcgccctgagcgccacactcccctgggcagggtccggagctctctggaagaagcccagcacctgcc

SEQ ID NO: 67 - DNA, 97 bp, synthetic construct
  misc_feature 1..97: note = Synthetic
  source 1..97: mol_type = other DNA
attttatagtgtgccatttagaagagacggagagggcccagaggacggtgccacattcacgtattagacgatagctcaagttgcccattcttcatat

SEQ ID NO: 68 - DNA, 128 bp, synthetic construct
  misc_feature 1..128: note = Synthetic
  source 1..128: mol_type = other DNA
gctccacaggcccagacattctagccccgaccgcctgtggcccccatcccaagaacccggggggctccgaggcttaccattggtccgcaggcccctccgtgccgggcacccacctccagctctggctg

SEQ ID NO: 69 - DNA, 110 bp, synthetic construct
  misc_feature 1..110: note = Synthetic
  source 1..110: mol_type = other DNA
tttcatgtatagaccaacagcaaagcctttactgccaagacttcctgtgtgcggcgccgctaccgtgagttcgtgtggctgagaaagcagctacagagaaatgctggttt

SEQ ID NO: 70 - DNA, 93 bp, synthetic construct
  misc_feature 1..93: note = Synthetic
  source 1..93: mol_type = other DNA
accctctctggctggactgttaaaagcaacgagaggccccggcacacctggaagctggccgcgaattcggcctctgggcctgtgtgtctgtga

SEQ ID NO: 71 - DNA, 115 bp, synthetic construct
  misc_feature 1..115: note = Synthetic
  source 1..115: mol_type = other DNA
tcttaaagaagtccccatgggccgcagcctggacgtctgctccgttctccaccaggagggtcaccagggccatgttgcgtctctcgatggcgatgtgcagtgctgtctggcctac

SEQ ID NO: 72 - DNA, 130 bp, synthetic construct
  misc_feature 1..130: note = Synthetic
  source 1..130: mol_type = other DNA
aggctgtccctttatttctcagaccggccgacacttagggaaaatagaaaagaacctatgtgaaatatcgggggtgaatttcacccgatatctggctgaatttcccccgatagttactaaagggagggaa

SEQ ID NO: 73 - DNA, 105 bp, synthetic construct
  misc_feature 1..105: note = Synthetic
  source 1..105: mol_type = other DNA
tttagtatgattttgagaattttgcgggtagttttggtttgtttttttttacgttttatcgcgatgtgcgtataatagtattgtgatttttgtggtgtattgtag

SEQ ID NO: 74 - DNA, 123 bp, synthetic construct
  misc_feature 1..123: note = Synthetic
  source 1..123: mol_type = other DNA
gttaagaaaccaacaggaaaaagaacgcacaactcccagcacagtgctggcgcctgtgaggcactcagccgacgggagctttgttcttcgttgtattgtggcggggaagcaacatggggcctt

SEQ ID NO: 75 - DNA, 98 bp, synthetic construct
  misc_feature 1..98: note = Synthetic
  source 1..98: mol_type = other DNA
ggggcattcctcaccacctcatccacgtagcggcagtgctggactgcgtcatagcgctcattctcgttcatcaccgtgaagcctttgaagttgtgtgt

SEQ ID NO: 76 - DNA, 139 bp, synthetic construct
  misc_feature 1..139: note = Synthetic
  source 1..139: mol_type = other DNA
ggagctgttgcagcagctcttcagaacgcctccacccgagcttctgcaaaccctgaccgcagtcgggggcattgggcagctcaccgctgctaaggaggagtctggtggccgaagccgtagtgggagtattgtggaactt

SEQ ID NO: 77 - DNA, 118 bp, synthetic construct
  misc_feature 1..118: note = Synthetic
  source 1..118: mol_type = other DNA
tctgagagctgtccacaacagggtcggcgtccaccaacacctcgatctccatggacttattgctcgtgaaggtcatgcgtcccgagatggtgatgacgcagcctgtggagaagggagg

SEQ ID NO: 78 - DNA, 109 bp, synthetic construct
  misc_feature 1..109: note = Synthetic
  source 1..109: mol_type = other DNA
tgaaggaaatgagagcaaaggtcgtagctgagtgcacgttgaaagaaagcgcggccatcaaccagatccttgggcggagggtaggtgacgcccttctcagggagaaggg

SEQ ID NO: 79 - DNA, 125 bp, synthetic construct
  misc_feature 1..125: note = Synthetic
  source 1..125: mol_type = other DNA
ttgccaggttaagcaatctgcaaacgttcgcatgacgtttatgcagccgagtcacatccacattctctccctgtcagttcctttcccgggagctagaaatattgaagtcatttaagtaggagatg

SEQ ID NO: 80 - DNA, 100 bp, synthetic construct
  misc_feature 1..100: note = Synthetic
  source 1..100: mol_type = other DNA
tgcaggcacctcagattggggcgtggcgaaacagattgcatttcctagaaggcccccagcgatgtggattgaagcgcaagagtgaggagttggataatca

SEQ ID NO: 81 - DNA, 119 bp, synthetic construct
  misc_feature 1..119: note = Synthetic
  source 1..119: mol_type = other DNA
tcctaagaagctcttgtgccaccatcgtgctgaaaagaagagaaagaagcgagtatatttctgacctcttgtgtggcgactgaattgtcggcctcggtctgcacccaggcacccccaga

SEQ ID NO: 82 - DNA, 121 bp, synthetic construct
  misc_feature 1..121: note = Synthetic
  source 1..121: mol_type = other DNA
gcctgctttgagatgtgagaaccgtcagtcgggcttggcgggggccgcgcctccatgctcccaggcagcacacattcctcagtctcgactctcgcccgttgtgagcctgcaaggatgccac

SEQ ID NO: 83 - DNA, 123 bp, synthetic construct
  misc_feature 1..123: note = Synthetic
  source 1..123: mol_type = other DNA
ggtgttacaggtagggcagagaacgcaagacccagcatctcggctctccagcactgccgccctagcacagggaggtggataacaagcaggcaaacgcacgatgaagaaaatgacaaatgctgg

SEQ ID NO: 84 - DNA, 135 bp, synthetic construct
  misc_feature 1..135: note = Synthetic
  source 1..135: mol_type = other DNA
agcacccccattgggttggaccgtgtgggacgccggcggcagatgagagcatccaacggtgagtctcagagtccctttcctttcagagctgctatgggcccatctgaccgtttccagctaacctgaggcagcagg

SEQ ID NO: 85 - DNA, 114 bp, synthetic construct
  misc_feature 1..114: note = Synthetic
  source 1..114: mol_type = other DNA
tgggccctgttgtcacagcccccgacttgcccagatttttccgccattgacgtcatggcggccggatgcgccgggcttcatcgacaccacggaggaagagaagagggcagatac

SEQ ID NO: 86 - DNA, 115 bp, synthetic construct
  misc_feature 1..115: note = Synthetic
  source 1..115: mol_type = other DNA
ctaggcttggatttcaactctgccgctgcatttcggtgaggccctgagatctctggtcttcaatttgcccttctacactgagcacggagaggcgtggagtagacaagggccaggg

(19) United States
(12) Patent Application Publication
(10) Pub. No.: US 2026/0265827 A1
(43) Pub. Date: Sep. 10, 2026

(54) CFDNA AS BIOMARKER FOR INFLAMMATORY BOWEL DISEASES

(71) Applicants: YISSUM RESEARCH DEVELOPMENT COMPANY OF THE HEBREW UNIVERSITY OF JERUSALEM LTD., Jerusalem (IL); SHAARE ZEDEK SCIENTIFIC LTD., Jerusalem (IL); HADASIT MEDICAL RESEARCH SERVICES & DEVELOPMENT LTD., Jerusalem (IL)

(72) Inventors: Yuval DOR, Jerusalem (IL); Eyal SHTEYER, Mevaseret Zion (IL); Ruth SHEMER, Mevasseret Zion (IL); Ami BEN YAAKOV, Jerusalem (IL); Esther MEYER ORLANSKI, Jerusalem (IL); Benjamin GLASER, Jerusalem (IL); Shari ORLANSKI, Jerusalem (IL); Bracha-Lea OCHANA, Jerusalem (IL)

(21) Appl. No.: 19/485,381

(22) PCT Filed: May 19, 2024
(86) PCT No.: PCT/IL2024/050493

§ 371 Date: Nov. 18, 2025

Related U.S. Application Data
(60) Provisional application No. 63/467,412, filed on May 18, 2023.

(51) Int. Cl. C12Q 1/6883

(52) U.S. Cl. CPC C12Q 1/6883; C12Q 2600/154

(57) ABSTRACT

Methods of diagnosing inflammatory bowel disease (IBD), ulcerative colitis (UC) or Crohn's disease (CD) in a subject in need thereof, comprising receiving a stool sample from the subject and measuring DNA levels in the stool sample are provided. Kits comprising at least one reagent that detects mammalian DNA and not bacterial DNA and at least one reagent that detects cell type-specific DNA methylation are also provided.

### CROSS REFERENCE TO RELATED APPLICATIONS

This application claims the benefit of priority of U.S. Provisional Patent Application No. 63/467,412, filed on May 18, 2023, the contents of which are all incorporated herein by reference in their entirety.

### REFERENCE TO AN ELECTRONIC SEQUENCE LISTING

The contents of the electronic sequence listing (HUJI-SHZ-P-0103-PCT.xml; Size: 111,550 bytes; and Date of Creation: May 14, 2024) is herein incorporated by reference in its entirety.

### FIELD OF INVENTION

The present invention is in the field of inflammatory bowel disease diagnostics.

### BACKGROUND OF THE INVENTION

The incidence and prevalence of inflammatory bowel diseases (IBD), including Crohn's disease (CD) and ulcerative colitis (UC), are rising worldwide, especially in children, and present numerous challenges. One significant challenge is the need for invasive follow-up with endoscopy due to insufficient non-invasive biomarkers. Fecal calprotectin is currently the most commonly used biomarker for IBD, both for diagnosis and follow-up. While it is a very sensitive marker and efficiently detects intestinal inflammation, it is not specific and may be elevated in various conditions. Thus, distinguishing new patients with IBD from patients with non-inflammatory conditions can be challenging and lead to unnecessary invasive endoscopic assessment. An additional diagnostic challenge is the scenario of high suspicion of CD with normal endoscopy, necessitating further tests, such as video capsule or MRI. Lastly, once the diagnosis is established there is not always correlation between clinical symptoms, blood and stool tests and actual bowel inflammation. Accurate non-invasive identification of patients with symptoms but with mucosal healing is critical in order to avoid costly and unnecessary invasive testing. Conversely, identification of those in clinical remission with underlying subclinical inflammation is an important treatment goal as understanding of the long-term implications of intestinal inflammation increases, and the ability to treat IBD improves.

Cell-free DNA (cfDNA) are nucleosome-sized fragments of DNA released from dying cells to blood and other tissues. An approach for detecting the tissue origins of cfDNA, using tissue-specific DNA methylation markers has been previously disclosed. Levels of tissue-specific cfDNA reflect the rate of cell death or turnover from a specific organ and can mirror tissue injury and disease. A new accurate in vitro test for diagnosing and characterizing inflammation of the bowel is greatly needed.

### SUMMARY OF THE INVENTION

The present invention provides methods of diagnosing inflammatory bowel disease (IBD), ulcerative colitis (UC) or Crohn's disease (CD) in a subject in need thereof, comprising receiving a stool sample from the subject and measuring DNA levels in the stool sample are provided. Kits comprising at least one reagent that detects mammalian DNA and not bacterial DNA and at least one reagent that detects cell type-specific DNA methylation are also provided.

According to a first aspect, there is provided a method of diagnosing inflammatory bowel disease (IBD) in a subject in need thereof, the method comprising receiving a stool sample from the subject and measuring DNA levels in said stool sample wherein a proportion of human DNA out of total DNA in the stool sample, or a proportion of leukocyte DNA, neutrophil DNA, eosinophil DNA or monocyte DNA out of total human DNA in the stool sample, or a combination thereof above a predetermined threshold indicates the subject suffers from IBD; thereby diagnosing IBD in a subject.

According to another aspect, there is provided a method of diagnosing inflammatory bowel disease (IBD) in a subject in need thereof, the method comprising receiving a stool sample from the subject and measuring DNA levels in the stool sample wherein a level of mammalian DNA, and specific leukocyte DNA above a predetermined threshold indicates the subject suffers from IBD; thereby diagnosing IBD in a subject.

According to another aspect, there is provided a method of diagnosing ulcerative colitis (UC) in a subject in need thereof, the method comprising receiving a stool sample from the subject and measuring DNA levels in the stool sample wherein a level of mammalian DNA, colon DNA, intestine DNA, leukocyte DNA, neutrophil DNA, monocyte

DNA, eosinophil DNA or B cell DNA above a predetermined threshold indicates the subject suffers from UC; thereby diagnosing UC in a subject.

According to another aspect, there is provided a method of diagnosing Crohn's Disease (CD) in a subject in need thereof, the method comprising receiving a stool sample from the subject and measuring mammalian DNA levels in the stool sample wherein a level of leukocyte DNA, neutrophil DNA, or eosinophil DNA above a predetermined threshold indicates the subject suffers from CD; thereby diagnosing CD in a subject.

According to some embodiments, IBD is Ulcerative colitis (UC), Crohn's Disease (CD) or both.

According to some embodiments, IBD is CD.

According to some embodiments, the predetermined threshold is a level of DNA in a stool sample from a healthy subject.

According to some embodiments, the predetermined threshold is a proportion of DNA in a stool sample from a healthy subject.

According to some embodiments, mammalian DNA is human DNA.

According to some embodiments, the diagnosing UC further comprises classifying the severity of UC in the subject, wherein total mammalian DNA, intestine DNA, or colon DNA above a predetermined threshold indicates the subject suffers from moderate or severe UC and wherein the predetermined threshold is a DNA level in a stool sample from a subject with mild UC.

According to some embodiments, the diagnosing UC or CD further comprises classifying the severity of UC or CD in the subject, wherein a proportion of human DNA, leukocyte DNA, or neutrophil DNA above a predetermined threshold indicates the subject suffers from moderate or severe UC or CD and wherein the predetermined threshold is a proportion of DNA in a stool sample from a subject with mild UC or CD.

According to some embodiments, the disease severity is as determined by a noninvasive index, optionally wherein the noninvasive index is selected from Pediatric Crohn's Disease Activity Index (wPCDAI) Mucosal Inflammation-Non-Invasive (MINI) index, and CD activity index (CDAI).

According to another aspect, there is provided a method of diagnosing IBD remission in a subject that suffered from IBD, the method comprising receiving a stool sample from the subject and measuring DNA levels in the stool sample wherein a level of total mammalian DNA, leukocyte DNA, colon DNA, intestine DNA, neutrophil DNA, monocyte DNA, eosinophil DNA, B cell DNA, T cell DNA, CD8 positive cell DNA or T regulatory cell (Treg) DNA below a predetermined threshold indicates the subject is in IBD remission; thereby diagnosing IBD remission in a subject.

According to another aspect, there is provided a method of diagnosing UC remission in a subject that suffered from UC, the method comprising receiving a stool sample from the subject and measuring DNA levels in the stool sample wherein a level of total mammalian DNA, leukocyte DNA, colon DNA, intestine DNA, neutrophil DNA, monocyte DNA, eosinophil DNA, B cell DNA, T cell DNA, CD8 positive cell DNA or T regulatory cell (Treg) DNA below a predetermined threshold indicates the subject is in UC remission; thereby diagnosing UC remission in a subject.

According to another aspect, there is provided a method of diagnosing CD remission in a subject that suffered from CD, the method comprising receiving a stool sample from the subject and measuring DNA levels in the stool sample wherein a level of total mammalian DNA, colon DNA, neutrophil DNA, or Treg DNA below a predetermined threshold indicates the subject is in CD remission; thereby diagnosing CD remission in a subject.

According to another aspect, there is provided a method of diagnosing IBD remission in a subject that suffered from IBD, the method comprising receiving a stool sample from said subject and measuring DNA levels in said stool sample wherein a proportion of human DNA out of total DNA in the stool sample, or a proportion of leukocyte DNA, colon DNA, or neutrophil cfDNA out of total human DNA in the stool sample below a predetermined threshold indicates the subject is in IBD remission; thereby diagnosing IBD remission in a subject.

According to some embodiments, the IBD is UC.

According to some embodiments, the predetermined threshold is a level of DNA in a stool sample from a subject suffering from active disease.

According to some embodiments, the predetermined threshold is a proportion of DNA in a stool sample from a subject suffering from active disease.

According to some embodiments, the subject is a child.

According to some embodiments, the method further comprises extracting DNA from the stool sample.

According to some embodiments, the method further comprises sequencing DNA from the stool sample.

According to some embodiments, DNA from a tissue or cell type that is not leukocytes comprises differential methylation of at least one locus as compared to methylation in leukocytes.

According to some embodiments, the locus comprises at least two CpG dinucleotides.

According to some embodiments, the locus comprises at least 5 CpG dinucleotides.

According to some embodiments, the locus is hypomethylated in the tissue or cell type and hypermethylated in leukocytes.

According to some embodiments, hypomethylation comprises methylation of less than 40% of CpG dinucleotides in the locus.

According to some embodiments, hypermethylation comprises methylation of more than 90% of CpG dinucleotides in the locus.

According to some embodiments, DNA from a tissue or cell type that is not leukocytes comprises differential methylation of the at least one locus as compared to at least 5 other tissues/cell types.

According to some embodiments, the locus is hypomethylated in the tissue or cell type and hypermethylated in the at least 5 other tissues/cell types.

According to some embodiments, hypermethylation comprises methylation of more than 80% of CpG dinucleotides in the locus.

According to some embodiments, the tissue or cell type that is not leukocytes and the at least 5 other tissues/cell types are selected from colon, intestine, neutrophils, monocytes, eosinophils, B cells, T cells, CD8 positive cells, and Tregs.

According to some embodiments, the tissue or cell type that is not leukocytes and the at least 5 other tissues/cell types are selected from colon, intestine, neutrophils, monocytes, eosinophils, B cells, T cells, CD8 positive cells, and Tregs and the at least one locus comprises differential methylation as compared to all other tissues or cell types.

According to some embodiments, the DNA is identified by a methylation specific assay or wherein the DNA is bisulfite converted.

According to some embodiments, the method further comprises sequencing the bisulfite converted DNA or amplifying the DNA with a nucleic acid molecule that hybridizes to the bisulfite converted DNA and not the unconverted DNA.

According to some embodiments, the mammalian DNA is identified by the presence of mammalian specific DNA sequence.

According to some embodiments, the human DNA is identified by the presence of human specific DNA sequence.

According to some embodiments, the mammalian specific DNA sequence is a primate or human specific DNA sequence, optionally wherein the primate or human specific DNA sequence is an Alu repeat.

According to some embodiments, the locus is selected from provided in FIG. 10.

According to some embodiments, the locus comprises a DNA sequence selected from SEQ ID NO: 59-86.

According to some embodiments, a combination of the proportion of human DNA and the proportion of neutrophil DNA above a predetermined threshold indicates the subject suffers from IBD.

According to some embodiments, the method further comprises treating a subject diagnosed with IBD, UC or CD or not in remission for IBD UC or CD with at least one anti-inflammatory agent.

According to some embodiments, the anti-inflammatory agent is selected from: steroids, an anti-metabolite agent, and dietary intervention.

According to some embodiments, the method further comprises performing a colonoscopy on a subject diagnosed with IBD, UC or CD.

According to some embodiments, the method further comprises discontinuing treatment of a subject diagnosed to be in remission.

According to some embodiments, the method further comprises not performing a colonoscopy on a subject diagnosed to be in remission.

According to some embodiments, the subject does not suffer from colorectal cancer. According to another aspect, there is provided a kit comprising:
a. at least one reagent that detects mammalian DNA and not bacterial DNA; and
b. at least one reagent that detects cell type or tissue-specific DNA methylation.

According to another aspect, there is provided a kit comprising:
a. at least one reagent that detects human DNA and not bacterial DNA; and
b. at least one reagent that detects cell type or tissue-specific DNA methylation.

According to some embodiments, mammalian DNA is primate or human DNA.

According to some embodiments, the reagent is sequence specific to a primate or human DNA sequence.

According to some embodiments, the reagent is sequence specific to a human DNA sequence.

According to some embodiments, the reagent that detects primate or human DNA is an Alu repeat specific probe or primer.

According to some embodiments, the cell type or tissue-specific DNA methylation is selected from leukocyte-specific DNA methylation, neutrophil-specific DNA methylation, eosinophil-specific DNA methylation, B cell-specific DNA methylation, colon-specific DNA methylation, intestine-specific DNA methylation and monocyte-specific DNA methylation.

According to some embodiments, the reagent that detect cell type or tissue-specific DNA methylation is a primer pair that amplifies a locus of at least 150 and at most 500 nucleotides that comprises at least two CpG dinucleotides that are uniquely methylated or unmethylated in the cell type or tissue.

According to some embodiments, the primer pair is selected from the sequences provided in SEQ ID NO: 3-58.

According to some embodiments, the kit further comprises at least one of: a reagent for extracting DNA from stool, a calibration curve for use of the reagents or instructions for use.

According to some embodiments, the kit is for use in a method of the invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1B: (1A) A flow chart describing the cohort of samples and experimental design. (1B) Table of the primers used to amplify the loci with colon or intestine specific hypomethylation.

FIGS. 2A-2J: Dot plots of total concentration of (2A) total cfDNA, (2B) colon cfDNA, (2C) intestine cfDNA, (2D) neutrophil cfDNA, (2E) monocyte cfDNA, (2F) eosinophil cfDNA, (2G) B cell cfDNA, (2H) T cell cfDNA, (2I) CD8 T cell cfDNA and (2J) T regulatory cells (Treg) cfDNA in plasma samples from healthy children, and patients with CD or UC. The fraction of cfDNA molecules from each cell type (averaged for markers used) was multiplied by the total concentration of cfDNA in each sample to obtain the concentration in genome equivalents per ml plasma. *, P<0.05.

FIGS. 3A-3D: Dot plots of percentage of human DNA (3A) in stool samples from control subjects and patients with active IBD or in remission, (3B) in stool of patients with CD or UC, (3C) in stool of patients with active or remitting UC or CD, and (3D) as a function of disease severity at the time of sampling. *, p<0.05; **, p<0.01; ***, p<0.001; *** p<0.0001. 3A-C uses Kolmogorov-Smirnov test. 3D uses Kruskal-Wallis test.

FIGS. 4A-4B: Dot plots of (4A) the percentage of small intestine, colon and leukocyte-derived DNA in the stool of patients with active or remitting IBD, and (4B) the percentage of tissue and immune cell-specific DNA in the stool of patients with active CD or UC. *, p<0.05; **, p<0.01; ***, p<0.001. Kolmogorov-Smirnov test.

FIGS. 5A-5D: Correlation matrix describing relationships between standard biomarkers and stool DNA markers (% human from total DNA and % of human DNA from indicated cell types) in patients with (5A) UC and (5B) CD. (5C-D) ROC curves showing (5C) the percentage of neutrophil DNA in stool (AUC=0.8484), and (5D) the percentage of neutrophil DNA in stool combined with the percentage of human DNA in stool (AUC=0.906) is able to distinguish active CD from healthy controls.

FIGS. 6A-6C: (6A-6B) Box and whisker plots of levels of biochemical biomarkers and stool DNA markers (median) in colonoscopy-verified patients (labeled as +) with (6A) UC (n=29) and (6B) CD (n=40). (6C) Plots showing the relative average contribution of DNA from different human tissues to each patient group.

FIGS. 7A-7D: (7A-B) Bar graphs of (7A) specificity of two novel pan-leukocyte methylation markers, (7B) validation of methylation markers specific for epithelial cells of the colon and small intestine, using genomic DNA extracted from the indicated sources. (7C-D) Spike-in experiments showing sensitivity of intestinal methylation markers. Genomic DNA of (7C) isolated colon or (7D) small intestine epithelial cells in the indicated amount was mixed with 3000 genome equivalents (10 ng) of leukocyte DNA, and respective methylation markers were applied to infer the presence of intestinal DNA.

FIGS. 8A-8J: Dot plots of the fraction of human DNA that is from (8A) intestine, (8B) colon, (8C), leukocytes, (8D) neutrophils, (8E) monocytes, (8F) eosinophils, (8G) B cells, (8H) T cells, (8I) CD8 T cells, and (8J) Tregs in the stool of UC patients as a function of disease severity.

FIGS. 9A-9J: Dot plots of the fraction of human DNA that is from (9A) intestine, (9B) colon, (9C), leukocytes, (9D) neutrophils, (9E) monocytes, (9F) eosinophils, (9G) B cells, (9H) T cells, (9I) CD9 T cells, and (9J) Tregs in the stool of CD patients as a function of disease severity.

FIG. 10: Table of primers and amplicons for various tissues and cell types.

### DETAILED DESCRIPTION OF THE INVENTION

The present invention, in some embodiments, provides methods of diagnosing inflammatory bowel disease (IBD), ulcerative colitis (UC) or Crohn's disease (CD) in a subject in need thereof, comprising receiving a stool sample from the subject and measuring DNA in the stool sample. Kits comprising at least one reagent that detects mammalian DNA and not bacterial DNA and at least one reagent that detects cell type-specific DNA methylation are also provided.

The invention is based, at least in part, on the development of a novel platform for minimally invasive assessment of the source of specific cell types in humans, based on DNA methylation patterns of cfDNA in body fluids. Children (0-18 years) were prospectively enrolled in various stages of disease. Stool samples were assessed for the proportion of human DNA present out of total DNA (most of the DNA is bacterial) and DNA with intestinal (duodenum and colon) and immunological markers (indicating an origin from those tissues/cells). 196 stool-derived DNA samples were collected from 122 children with Crohn's disease (CD), 74 with ulcerative colitis (UC) and 40 controls.

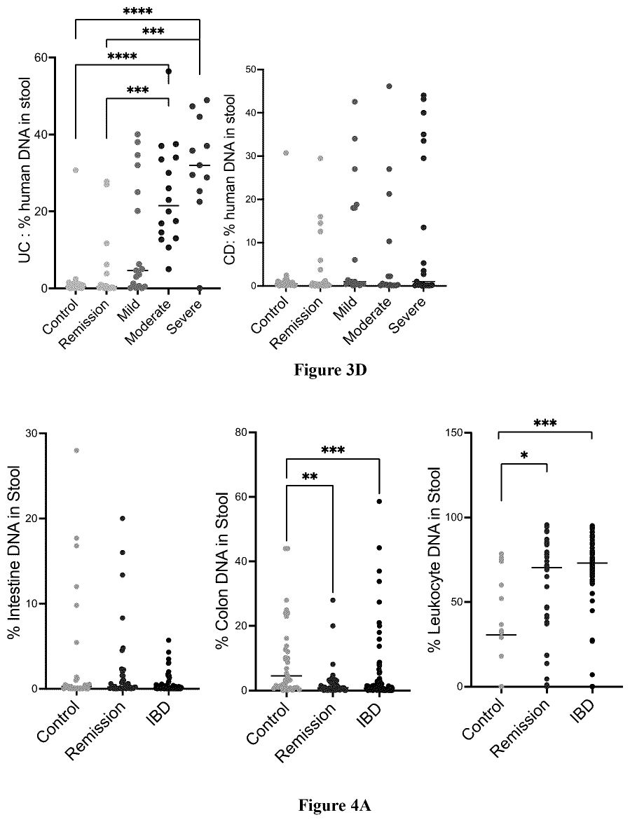

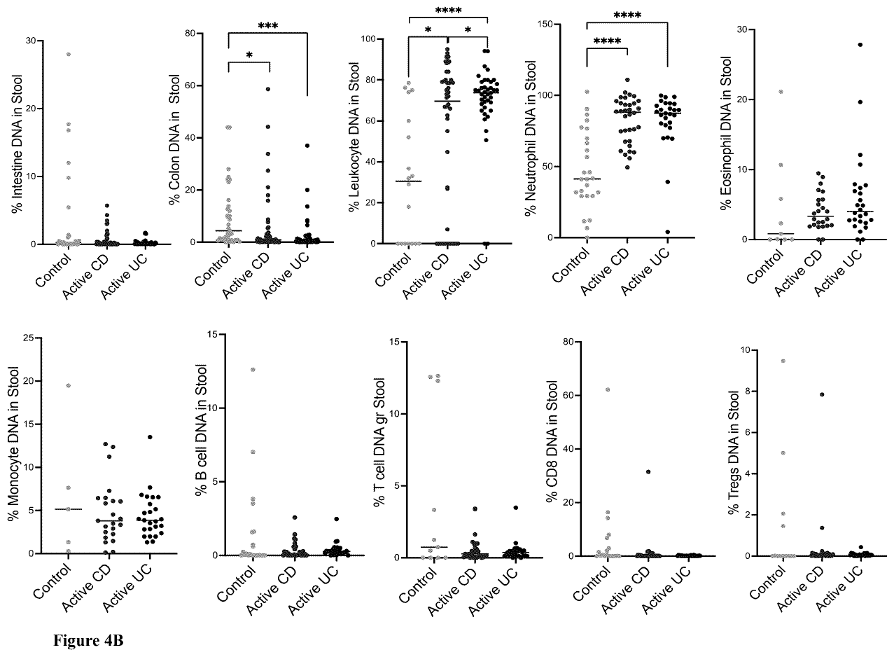

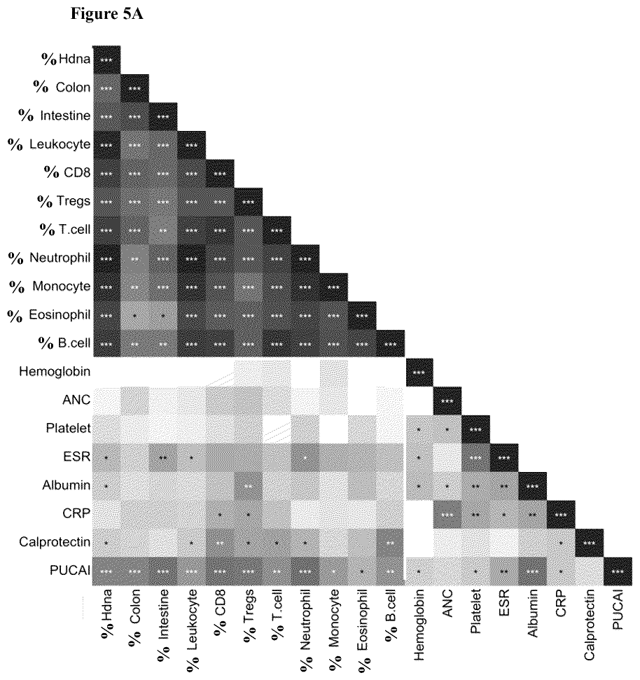

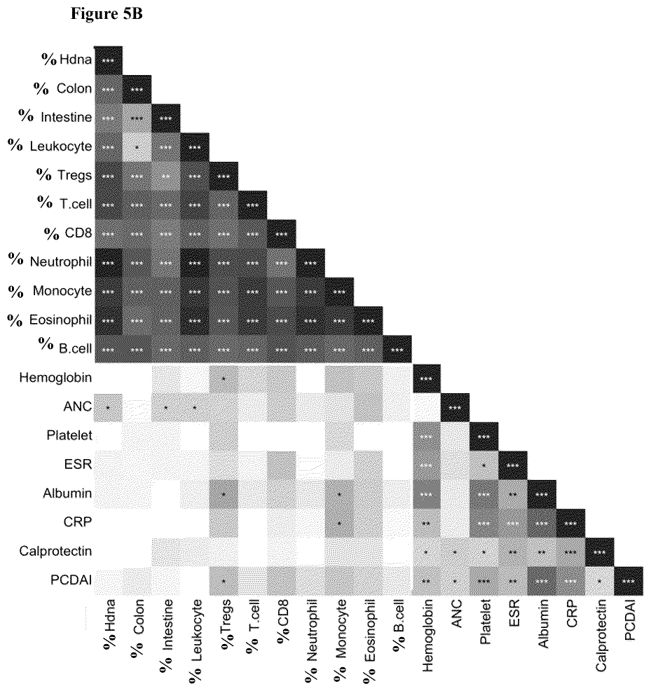

The proportion of human DNA (FIG. 3) and DNA with intestine and leukocyte markers (FIG. 4) differentiated between controls and IBD (CD and UC). Proportion of DNA correlated with clinical disease activity in UC (FIG. 3D) but not in CD. Children in remission had similar DNA levels as in controls. The use of DNA was highly predictive for determining IBD, UC and CD. Thus, in stool, human DNA and DNA from specific tissues/cells can differentiate between controls and children with IBD; and in UC, DNA correlates with disease activity.

In total it was surprisingly found that stool samples from IBD patients contained elevated proportions of human DNA compared with controls, which was driven by an increased presence of neutrophil DNA. The fraction of human DNA in stool and its methylation patterns correlated with clinical disease activity in both ulcerative colitis (UC) and Crohn's disease (CD). Patients in remission displayed normal concentration and methylation patterns of human DNA in stool. The correlation between disease state and stool DNA was stronger in the sub-group of patients with colonoscopy-verified pathology, supporting the validity of the assay.

By a first aspect, there is provided a method of diagnosing inflammatory bowel disease (IBD) in a subject, the method comprising receiving DNA levels in a stool sample from the subject, wherein levels of DNA above a predetermined threshold indicate the subject suffers from IBD, thereby diagnosing IBD in a subject.

By another aspect, there is provided a method of diagnosing a subject as not suffering from inflammatory bowel disease (IBD), the method comprising receiving DNA levels in a stool sample from the subject, wherein levels of DNA below a predetermined threshold indicate the subject does not suffer from IBD, thereby diagnosing IBD in a subject.

By another aspect, there is provided a method of diagnosing ulcerative colitis (UC) in a subject, the method comprising receiving DNA levels in a stool sample from the subject, wherein levels of DNA above a predetermined threshold indicate the subject suffers from UC, thereby diagnosing UC in a subject.

By another aspect, there is provided a method of diagnosing a subject as not suffering from UC, the method comprising receiving DNA levels in a stool sample from the subject, wherein levels of DNA below a predetermined threshold indicate the subject does not suffer from UC, thereby diagnosing UC in a subject.

By another aspect, there is provided a method of diagnosing Crohn's Disease (CD) in a subject, the method comprising receiving DNA levels in a stool sample from the subject, wherein levels of DNA above a predetermined threshold indicate the subject suffers from CD, thereby diagnosing CD in a subject.

By another aspect, there is provided a method of diagnosing a subject as not suffering from CD, the method comprising receiving DNA levels in a stool sample from the subject, wherein levels of DNA below a predetermined threshold indicate the subject does not suffer from CD, thereby diagnosing CD in a subject.

By another aspect, there is provided a method of diagnosing IBD remission in a subject the method comprising receiving DNA levels in a stool sample from the subject, wherein levels of DNA below a predetermined threshold indicate the subject is in IBD remission, thereby diagnosing IBD remission in a subject.

By another aspect, there is provided a method of diagnosing IBD active disease in a subject the method comprising receiving DNA levels in a stool sample from the subject, wherein levels of DNA above a predetermined threshold indicate the subject suffers from active IBD, thereby diagnosing active IBD in a subject.

By another aspect, there is provided a method of diagnosing UC remission in a subject the method comprising receiving DNA levels in a stool sample from the subject, wherein levels of DNA below a predetermined threshold indicate the subject is in UC remission, thereby diagnosing UC remission in a subject.

By another aspect, there is provided a method of diagnosing UC active disease in a subject the method comprising receiving DNA levels in a stool sample from the subject, wherein levels of DNA above a predetermined threshold indicate the subject suffers from active UC, thereby diagnosing active UC in a subject.

By another aspect, there is provided a method of diagnosing CD remission in a subject the method comprising receiving DNA levels in a stool sample from the subject, wherein levels of DNA below a predetermined threshold indicate the subject is in CD remission, thereby diagnosing CD remission in a subject.

By another aspect, there is provided a method of diagnosing CD active disease in a subject the method comprising receiving DNA levels in a stool sample from the subject, wherein levels of DNA above a predetermined threshold indicate the subject suffers from active CD, thereby diagnosing active CD in a subject.

In some embodiments, the method is a diagnostic method. In some embodiments, the method is a prognostic method. In some embodiments, the method is an in vitro method. In some embodiments, the method is an ex vivo method. In some embodiments, the method is a method of liquid biopsy. In some embodiments, the method is not a method of diagnosing colorectal cancer. In some embodiments, the method is a method of diagnosing IBD, CD or UC. In some embodiments, the method is a method of diagnosing IBD, CD and UC. In some embodiments, IBD comprises UC or CD. In some embodiments, the method is a method of differentiating between UC and CD. In some embodiments, diagnosing is monitoring. In some embodiments, diagnosing comprises monitoring.

As used herein, the term “IBD” is used to refer to a general inflammatory condition characterized by chronic inflammation of the gastrointestinal tract. In some embodiments, IBD comprises colitis and CD. As used herein, the term “UC” refers to a chronic condition characterized by inflammation and ulcers on the inner lining of the large intestine. In some embodiments, colitis is ulcerative colitis. In some embodiments, ulcerative colitis is autoimmune ulcerative colitis. As used herein, the term “CD” refers to a chronic inflammatory condition characterized by swelling of the tissue of the digestive tract. In some embodiments, IBD is UC. In some embodiments, IBD is CD.

In some embodiments, the subject is a mammal. In some embodiments, the mammal is a primate. In some embodiments, the primate is a human. In some embodiments, the subject is in need of a method of the invention. In some embodiments, the subject is at risk for developing IBD, CD or UC. In some embodiments, the subject is suspected of suffering from IBD, CD or UC. In some embodiments, the subject has symptoms of IBD, CD or UC. Examples of symptoms include, but are not limited to, abdominal pain, diarrhea, fatigue, weight loss and malnutrition. In some embodiments, the subject is a child. In some embodiments, a child is a subject younger than 18 years of age. In some embodiments, a child is a subject that is 18 years of age or younger. In some embodiments, a child is a subject younger than 21, 20, 19, 18, 17, 16, 15, 14, 13, 12, 11, 10, 9, 8, 7 or 6 years of age. Each possibility represents a separate embodiment of the invention. In some embodiments, a child is a subject younger than 13 years of age.

In some embodiments, the method comprises receiving a stool sample. In some embodiments, the sample is from the subject. In some embodiments, the method comprises extracting DNA from the stool sample. In some embodiments, the method comprises amplifying DNA from the stool sample. In some embodiments, the method comprises sequencing DNA from the stool sample. In some embodiments, sequencing is next generation sequencing. In some embodiments, sequencing is high throughput sequencing. In some embodiments, sequencing is massively parallel sequencing.

In some embodiments, the DNA is cell free DNA (cfDNA). In some embodiments, the method does not comprise or is devoid of lysing cells from the stool sample. In some embodiments, the method comprises lysing cells from the stool sample. In some embodiments, the cells in the stool sample are microbial cells. In some embodiments, the microbial cells are bacterial cells. In some embodiments, the method comprises using a kit designed to extract bacterial DNA. For example, the Qiagen Dneasy powerLyzer power Soil Kit was used herein. As the total percentage of human DNA from within both human and bacterial DNA is being measured, it is important that bacterial DNA be isolated. As such, kits designed to extract human DNA from feces without lysing bacterial cells would not be suitable for this purpose.

In some embodiments, the method comprises measuring DNA in the sample. In some embodiments, measuring DNA is measuring DNA levels. In some embodiments, DNA is total cfDNA. In some embodiments, total DNA is total mammalian DNA. In some embodiments, DNA is mammalian DNA. In some embodiments, DNA is not bacterial DNA. In some embodiments, mammalian DNA is non-bacterial DNA. In some embodiments, mammalian DNA is primate DNA. In some embodiments, mammalian DNA is human DNA. In some embodiments, measuring DNA is measuring the percentage of mammalian DNA within the total DNA. It will be understood by a skilled artisan that stool samples will contain DNA from bacteria; thus, the mammalian/human DNA can be measured as a percentage of all DNA present in the sample. In some embodiments, the method comprises measuring all DNA in the sample and measuring mammalian DNA in the sample and calculating the percentage of mammalian DNA out of all the DNA in the sample. In some embodiments, the method comprises calculating the percentage of all DNA that is mammalian DNA.

In some embodiments, the total amount of mammalian DNA is measured. In some embodiments, the total level of mammalian DNA is measured. In some embodiments, the total amount of mammalian DNA is received. In some embodiments, the total level of mammalian DNA is received. In some embodiments, the percentage of mammalian DNA is measured. In some embodiments, the percentage of mammalian DNA is received. In some embodiments, a total level or amount of mammalian DNA above a predetermined threshold indicates the subject suffers from IBD. In some embodiments, a total level or amount of mammalian DNA above a predetermined threshold indicates the subject suffers from UC. In some embodiments, a total level or amount of mammalian DNA above a predetermined threshold indicates the subject suffers from CD. In some embodiments, a percentage of mammalian DNA above a predetermined threshold indicates the subject suffers from IBD. In some embodiments, a percentage of mammalian DNA above a predetermined threshold indicates the subject suffers from UC. In some embodiments, a percentage of DNA above a predetermined threshold indicates the subject suffers from CD. In some embodiments, a percentage of total DNA that is mammalian DNA above a predetermined threshold indicates the subject suffers from IBD. In some embodiments, a percentage of total DNA that is mammalian DNA above a predetermined threshold indicates the subject suffers from UC. In some embodiments, a percentage of total DNA that is mammalian DNA above a predetermined threshold indicates the subject suffers from CD.

In some embodiments, above is significantly above. In some embodiments, significantly is statistically significantly. In some embodiments, above is at least above by 5, 10, 15, 20, 25, 30, 35, 40, 45, 50, 55, 60, 65, 70, 75, 80, 85, 90, 95, 100, 125, 150, 175, 200, 250, 300, 350, 400, 450, or 500%. Each possibility represents a separate embodiment of the invention.

Methods of measuring mammalian or human DNA are well known in the art and any such method may be used so long as it does not also detect/measure bacterial DNA. In some embodiments, the method of measuring mammalian DNA is a method of measuring mammalian cfDNA. In some embodiments, mammalian cfDNA is human cfDNA. It will be understood that while there will be intact bacterial cells in the feces there will not be (for the most part) intact human cells, thus the human DNA will be cfDNA while the bacterial DNA may be a mix of cellular and cell free DNA. In some embodiments, the measuring comprises detecting a mammalian-specific DNA sequence. In some embodiments, the measuring is sequence specific. In some embodiments, measuring comprises amplifying a human specific genetic sequence. In some embodiments, measuring comprises quantifying a human specific genetic sequence. In some embodiments, measuring comprises detecting a human specific genetic sequence. In some embodiments, the measuring comprises detecting mammalian specific DNA methylation. In some embodiments, the measuring comprises detecting an Alu repeat sequence. In some embodiments, an Alu repeat is an Alu element. In some embodiments, a mammalian-specific DNA sequence is a primate-specific DNA sequence. In some embodiments, a primate-specific DNA sequence is a human-specific DNA sequence. Alu repeats are well known in that they are found in the genome of primates and particularly humans. They can be used to detect cfDNA that is specifically from humans and not bacteria. Alu sequences, probes and primers are known in the art and can be purchased for example from BioGenex. Alu repeats are just one example of such useful sequences, however many other primate or even human specific sequences are known in the art. Further, human specific genetic sequence, such as the fragment of the gene SFPTC-1, described hereinbelow, can be used. There are thousands of specific sequences such as this that can be used for detecting and quantifying human DNA.

In some embodiments, DNA is leukocyte DNA. In some embodiments, leukocyte DNA levels are measured. In some embodiments, leukocyte DNA levels are received. In some embodiments, leukocyte levels are total leukocyte levels. In some embodiments, the percentage of human DNA that is leukocyte DNA is measured. In some embodiments, the percentage of human DNA that is leukocyte DNA is calculated. In some embodiments, the percentage of human DNA that is leukocyte DNA is received. In some embodiments, percentage is proportion. In some embodiments, leukocyte levels do not comprise the methylation status or activity in leukocytes, but rather is the total amount of leukocyte DNA present in the sample. In some embodiments, the method comprises identifying a DNA molecule as originating from leukocytes. In some embodiments, the identifying comprises detecting in the DNA molecule a DNA methylation pattern indicative of leukocytes. In some embodiments, primers for the detection of leukocyte specific DNA methylation are selected from SEQ ID NO: 21 and 22 and SEQ ID NO: 23 and 24. In some embodiments, the DNA sequence is selected from SEQ ID NO: 68 and 69. In some embodiments, the percentage of the mammalian DNA that is leukocyte DNA is calculated. In some embodiments, the method comprises calculating the percentage of mammalian DNA that is leukocyte DNA.

In some embodiments, DNA is neutrophil DNA. In some embodiments, neutrophil DNA levels are measured. In some embodiments, neutrophil DNA levels are received. In some embodiments, neutrophil levels are total neutrophil levels. In some embodiments, the percentage of human DNA that is neutrophil DNA is measured. In some embodiments, the percentage of human DNA that is neutrophil DNA is calculated. In some embodiments, the percentage of human DNA that is neutrophil DNA is received. In some embodiments, the method comprises identifying a DNA molecule as originating from neutrophil. In some embodiments, the identifying comprises detecting in the DNA molecule a DNA methylation pattern indicative of neutrophil. In some embodiments, primers for the detection of neutrophil specific DNA methylation are selected from SEQ ID NO: 45 and 46, SEQ ID NO: 47 and 48 and SEQ ID NO: 49 and 50. In some embodiments, the DNA sequence is selected from SEQ ID NO: 80, 81 and 82. In some embodiments, the percentage of the mammalian DNA that is neutrophil DNA is calculated. In some embodiments, the method comprises calculating the percentage of mammalian DNA that is neutrophil DNA.

In some embodiments, DNA is eosinophil DNA. In some embodiments, eosinophil DNA levels are measured. In some embodiments, eosinophil DNA levels are received. In some embodiments, eosinophil levels are total eosinophil levels. In some embodiments, the percentage of human DNA that is eosinophil DNA is measured. In some embodiments, the percentage of human DNA that is eosinophil DNA is calculated. In some embodiments, the percentage of human DNA that is eosinophil DNA is received. In some embodiments, the method comprises identifying a DNA molecule as originating from eosinophil. In some embodiments, the identifying comprises detecting in the DNA molecule a DNA methylation pattern indicative of eosinophil. In some embodiments, primers for the detection of eosinophil specific DNA methylation are selected from SEQ ID NO: 35 and 36, SEQ ID NO: 37 and 38 and SEQ ID NO: 39 and 40. In some embodiments, the DNA sequence is selected from SEQ ID NO: 75, 76 and 77. In some embodiments, the percentage of the mammalian DNA that is eosinophil DNA is calculated. In some embodiments, the method comprises calculating the percentage of mammalian DNA that is eosinophil DNA.

In some embodiments, cfDNA is B cell DNA. In some embodiments, B cell DNA levels are measured. In some embodiments, B cell DNA levels are received. In some embodiments, B cell levels are total B cell levels. In some embodiments, the method comprises identifying a DNA molecule as originating from B cell. In some embodiments, the percentage of human DNA that is B cell DNA is measured. In some embodiments, the percentage of human DNA that is B cell DNA is calculated. In some embodiments, the percentage of human DNA that is B cell DNA is received. In some embodiments, the identifying comprises detecting in the DNA molecule a DNA methylation pattern indicative of B cell. In some embodiments, primers for the detection of B cell specific DNA methylation are selected from SEQ ID NO: 25 and 26, SEQ ID NO: 27 and 28 and SEQ ID NO: 29 and 30. In some embodiments, the DNA sequence is selected from SEQ ID NO: 70, 71 and 72. In some embodiments, the percentage of the mammalian DNA that is B cell DNA is calculated. In some embodiments, the method comprises calculating the percentage of mammalian DNA that is B cell DNA.

In some embodiments, DNA is colon DNA. In some embodiments, colon DNA levels are measured. In some embodiments, colon DNA levels are received. In some embodiments, colon levels are total colon levels. In some embodiments, the percentage of human DNA that is colon DNA is measured. In some embodiments, the percentage of human DNA that is colon DNA is calculated. In some embodiments, the percentage of human DNA that is colon DNA is received. In some embodiments, the method comprises identifying a DNA molecule as originating from colon. In some embodiments, the identifying comprises detecting in the DNA molecule a DNA methylation pattern indicative of colon. In some embodiments, primers for the detection of colon specific DNA methylation are selected from SEQ ID NO: 3 and 4, SEQ ID NO: 5 and 6, SEQ ID NO: 7 and 8, SEQ ID NO: 9 and SEQ ID NO: 10, SEQ ID NO: 11 and SEQ ID NO: 12, and SEQ ID NO: 13 and SEQ ID NO: 14. In some embodiments, the DNA sequence is selected from SEQ ID NO: 59, 60, 61, 62, 63 and 64. In some embodiments, the percentage of the mammalian DNA that is colon DNA is calculated. In some embodiments, the method comprises calculating the percentage of mammalian DNA that is colon DNA.

In some embodiments, DNA is intestine DNA. In some embodiments, intestine DNA levels are measured. In some embodiments, intestine DNA levels are received. In some embodiments, intestine levels are total intestine levels. In some embodiments, the percentage of human DNA that is intestine DNA is measured. In some embodiments, the percentage of human DNA that is intestine DNA is calculated. In some embodiments, the percentage of human DNA that is intestine DNA is received. In some embodiments, the method comprises identifying a DNA molecule as originating from intestine. In some embodiments, the identifying comprises detecting in the DNA molecule a DNA methylation pattern indicative of intestine. In some embodiments, primers for the detection of intestine specific DNA methylation are selected from SEQ ID NO: 15 and 16, SEQ ID NO: 17 and 18 and SEQ ID NO: 19 and 20. In some embodiments, the DNA sequence is selected from SEQ ID NO: 65, 66 and 67. In some embodiments, the percentage of the mammalian DNA that is intestine DNA is calculated. In some embodiments, the method comprises calculating the percentage of mammalian DNA that is intestine DNA.

In some embodiments, DNA is monocyte DNA. In some embodiments, monocyte DNA levels are measured. In some embodiments, monocyte DNA levels are received. In some embodiments, monocyte levels are total monocyte levels. In some embodiments, the percentage of human DNA that is monocyte DNA is measured. In some embodiments, the percentage of human DNA that is monocyte DNA is calculated. In some embodiments, the percentage of human DNA that is monocyte DNA is received. In some embodiments, the method comprises identifying a DNA molecule as originating from monocyte. In some embodiments, the identifying comprises detecting in the DNA molecule a DNA methylation pattern indicative of monocyte. In some embodiments, primers for the detection of monocyte specific DNA methylation are selected from SEQ ID NO: 41 and 42, and SEQ ID NO: 43 and 44. In some embodiments, the DNA sequence is selected from SEQ ID NO: 78 and 79. In some embodiments, the percentage of the mammalian DNA that is monocyte DNA is calculated. In some embodiments, the method comprises calculating the percentage of mammalian DNA that is monocyte DNA.

In some embodiments, DNA is T cell DNA. In some embodiments, T cell DNA levels are measured. In some embodiments, T cell DNA levels are received. In some embodiments, T cell levels are total T cell levels. In some embodiments, the percentage of human DNA that is T cell DNA is measured. In some embodiments, the percentage of human DNA that is T cell DNA is calculated. In some embodiments, the percentage of human DNA that is T cell DNA is received. In some embodiments, the method comprises identifying a DNA molecule as originating from T cell. In some embodiments, the identifying comprises detecting in the DNA molecule a DNA methylation pattern indicative of T cell. In some embodiments, primers for the detection of T cell specific DNA methylation are selected from SEQ ID NO: 51 and 52, and SEQ ID NO: 53 and 54. In some embodiments, the DNA sequence is selected from SEQ ID NO: 83 and 84. In some embodiments, the percentage of the mammalian DNA that is T cell DNA is calculated. In some embodiments, the method comprises calculating the percentage of mammalian DNA that is T cell DNA.

In some embodiments, DNA is CD8 positive cell DNA. In some embodiments, CD8 positive cells is CD8 positive T cells In some embodiments, CD8 positive cell DNA levels are measured. In some embodiments, CD8 positive cell DNA levels are received. In some embodiments, CD8 positive cell levels are total CD8 positive cell levels. In some embodiments, the percentage of human DNA that is CD8 positive cell DNA is measured. In some embodiments, the percentage of human DNA that is CD8 positive cell DNA is calculated. In some embodiments, the percentage of human DNA that is CD8 positive cell

DNA is received. In some embodiments, the method comprises identifying a DNA molecule as originating from CD8 positive cell. In some embodiments, the identifying comprises detecting in the DNA molecule a DNA methylation pattern indicative of CD8 positive cell. In some embodiments, primers for the detection of CD8 positive cell specific DNA methylation are selected from SEQ ID NO: 31 and 32, and SEQ ID NO: 33 and 34. In some embodiments, the DNA sequence is selected from SEQ ID NO: 73 and 74. In some embodiments, the percentage of the mammalian DNA that is CD8 positive cell DNA is calculated. In some embodiments, the method comprises calculating the percentage of mammalian DNA that is CD8 positive cell DNA.

In some embodiments, DNA is T regulatory cell (Treg) DNA. In some embodiments, Treg DNA levels are measured. In some embodiments, Treg DNA levels are received. In some embodiments, Treg levels are total Treg levels. In some embodiments, the percentage of human DNA that is Treg DNA is measured. In some embodiments, the percentage of human DNA that is Treg DNA is calculated. In some embodiments, the percentage of human DNA that is Treg DNA is received. In some embodiments, the method comprises identifying a DNA molecule as originating from Treg. In some embodiments, the identifying comprises detecting in the DNA molecule a DNA methylation pattern indicative of Treg. In some embodiments, primers for the detection of Treg specific DNA methylation are selected from SEQ ID NO: 55 and 56, and SEQ ID NO: 57 and 58. In some embodiments, the DNA sequence is selected from SEQ ID NO: 85 and 86. In some embodiments, the percentage of the mammalian DNA that is Treg DNA is calculated. In some embodiments, the method comprises calculating the percentage of mammalian DNA that is Treg DNA.

In some embodiments, the disease is IBD and the levels are levels of mammalian DNA. In some embodiments, the disease is IBD and the levels are levels of leukocyte DNA. In some embodiments, the disease is IBD and the levels are levels of colon DNA. In some embodiments, the disease is IBD and the levels are levels of intestine DNA. In some embodiments, the disease is IBD and the levels are levels of neutrophil DNA. In some embodiments, the disease is IBD and the levels are levels of eosinophil DNA. In some embodiments, the disease is IBD and the levels are levels of B cell DNA. In some embodiments, IBD is IBD remission and the levels are levels of mammalian DNA. In some embodiments, IBD is IBD remission and the levels are levels of leukocyte DNA. In some embodiments, IBD is IBD remission and the levels are levels of colon DNA. In some embodiments, IBD is IBD remission and the levels are levels of intestine DNA. In some embodiments, IBD is IBD remission and the levels are levels of neutrophil DNA. In some embodiments, IBD is IBD remission and the levels are levels of monocyte DNA. In some embodiments, IBD is IBD remission and the levels are levels of eosinophil DNA. In some embodiments, IBD is IBD remission and the levels are levels of B cell DNA. In some embodiments, IBD is IBD remission and the levels are levels of T cell DNA. In some embodiments, IBD is IBD remission and the levels are levels of CD8 positive cell DNA. In some embodiments, IBD is IBD remission and the levels are levels of Treg DNA. In some embodiments, levels are relative levels. In some embodiments, levels of mammalian DNA are relative to all DNA in the sample. In some embodiments, levels of cell type specific DNA are relative to all mammalian DNA in the sample.

In some embodiments, the disease is UC and the levels are levels of mammalian DNA. In some embodiments, the disease is UC and the levels are levels of colon DNA. In some embodiments, the disease is UC and the levels are levels of intestine DNA. In some embodiments, the disease is UC and the levels are levels of leukocyte DNA. In some embodiments, the disease is UC and the levels are levels of neutrophil DNA. In some embodiments, the disease is UC and the levels are levels of monocyte DNA. In some embodiments, the disease is UC and the levels are levels of eosinophil DNA. In some embodiments, the disease is UC and the levels are levels of B cell DNA. In some embodiments, UC is UC remission and the levels are levels of mammalian DNA. In some embodiments, UC is UC remission and the levels are levels of leukocyte DNA. In some embodiments, UC is UC remission and the levels are levels of colon DNA. In some embodiments, UC is UC remission and the levels are levels of intestine DNA. In some embodiments, UC is UC remission and the levels are levels of neutrophil DNA. In some embodiments, UC is UC remission and the levels are levels of monocyte DNA. In some embodiments, UC is UC remission and the levels are levels of eosinophil DNA. In some embodiments, UC is UC remission and the levels are levels of B cell DNA. In some embodiments, UC is UC remission and the levels are levels of T cell DNA. In some embodiments, UC is UC remission and the levels are levels of CD8 positive cell DNA. In some embodiments, UC is UC remission and the levels are levels of Treg DNA.

In some embodiments, the disease is CD and the levels are levels of mammalian DNA. In some embodiments, the disease is CD and the levels are levels of leukocyte DNA. In some embodiments, the disease is CD and the levels are levels of colon DNA. In some embodiments, the disease is CD and the levels are levels of intestine DNA. In some embodiments, the disease is CD and the levels are levels of neutrophil DNA. In some embodiments, the disease is CD and the levels are levels of eosinophil DNA. In some embodiments, CD is CD remission and the levels are levels of total mammalian DNA. In some embodiments, CD is CD remission and the levels are levels of colon DNA. In some embodiments, CD is CD remission and the levels are levels of neutrophil DNA. In some embodiments, CD is CD remission and the levels are levels of Treg DNA.

In some embodiments, a combination of DNAs is measured. In some embodiments, human DNA and a cell type or tissue specific DNA is measured. In some embodiments, the proportion of human DNA and the proportion of a cell type or tissue specific DNA is measured. In some embodiments, the proportion of two different cell type or tissue specific DNAs is measured. In some embodiments, a combination of DNA above a predetermined threshold is indicative of IBD. In some embodiments, a combination of DNA below a predetermined threshold is indicative of remission. In some embodiments, the combination is combination of human DNA proportion and neutrophil proportion. In some embodiments, the combination is combination of human DNA proportion and leukocyte proportion. In some embodiments, the combination is combination of human DNA proportion and eosinophil proportion. In some embodiments, the combination is combination of human DNA proportion and monocyte proportion. In some embodiments, the combination is combination of two different cell type or tissue specific DNAs. In some embodiments, the combination is proportion of leukocyte DNA and proportion of neutrophil DNA. In some embodiments, the combination is proportion of leukocyte DNA and proportion of eosinophil DNA. In some embodiments, the combination is proportion of leukocyte DNA and proportion of monocyte DNA. In some embodiments, the combination is proportion of eosinophil DNA and proportion of neutrophil DNA. In some embodiments, the combination is proportion of monocyte DNA and proportion of neutrophil DNA. In some embodiments, the combination is proportion of eosinophil DNA and proportion of monocyte DNA.

In some embodiments, cell type or tissue specific DNA is determined by a methylation pattern in the DNA. In some embodiments, a methylation pattern in the DNA is identified. In some embodiments, a DNA is assigned as originating in a cell type or tissue if it possesses a methylation pattern at a locus indicative of the cell type of tissue. In some embodiments, the pattern is the methylation status of at least 2 CpG dinucleotides in the locus. In some embodiments, the locus comprises at least 2 CpG dinucleotides. In some embodiments, the locus comprises or consists of at least 150 nucleotides. In some embodiments, the locus comprises or consists of at least 200 nucleotides. In some embodiments, the locus comprises or consists of at most 500 nucleotides. In some embodiments, the locus comprises or consists of between 150 and 500 nucleotides. In some embodiments, the locus comprises or consists of between 200 and 500 nucleotides. In some embodiments, at least 2 is at least 3. In some embodiments, at least 2 is at least 4. In some embodiments, at least 2 is 2 to 4. In some embodiments, at least 2 is 3 to 4. In some embodiments, at least 2 is at least 5.

In some embodiments, the locus is an informative locus. As used herein, the terms “informative locus” and “informative genomic locus” are used synonymously and refer to a unique DNA sequence in a particular location in the genome that when associated with a particular methylation pattern is informative of the tissue or cell type of origin of the DNA sequence. In some embodiments, the pattern is hypomethylation. In some embodiments, all CpGs of the locus are demethylated in the tissue or cell type of origin. In some embodiments, the pattern is hypermethylation. In some embodiments, all the CpGs of the locus are methylated in the tissue or cell type of origin.

In some embodiments, a DNA from a tissue or cell type that is not leukocytes comprises differential methylation of at least one locus as compared to methylation in leukocyte. In some embodiments, a DNA from a tissue or cell type that is not leukocytes comprises a locus with differential methylation as compared the locus's methylation in leukocytes. In some embodiments, the locus is hypomethylated as compared to leukocytes. In some embodiments, the locus is hypermethylated as compared to leukocytes. In some embodiments, the locus is hypomethylated in the tissue or cell type and hypermethylated in leukocytes. In some embodiments, the locus is hypermethylated in the tissue or cell type and hypomethylated in leukocytes.

In some embodiments, a DNA from leukocytes comprises differential methylation of at least one locus as compared to at least two other non-leukocyte tissues or cell types. In some embodiments, a DNA from leukocytes comprises a locus with differential methylation as compared to at least two other non-leukocyte tissues or cell types. In some embodiments, at least 2 is at least 2, 3, 4, 5, 6, 7, 8, 9, or 10. Each possibility represents a separate embodiment of the invention. In some embodiments, at least 2 is all other non-leukocyte tissues or cell types. In some embodiments, all other non-leukocyte tissues or cell types is all other non-leukocyte tissues or cell types measured. In some embodiments, a DNA from a tissue of cell type is determined by differential methylation of at least one locus as compared to at least 1, 2, 3, 4, 5, 6, 7, 8, 9, or 10 other tissues or cell types. Each possibility represents a separate embodiment of the invention. In some embodiments, a DNA from a tissue of cell type is determined by differential methylation of at least one locus as compared to at least 5 other tissues or cell types. In some embodiments, a locus is considered informative if it comprises differential methylation for a tissue or cell type as compared to at least 5 other tissues or cell types. In some embodiments, at least 5 is all other tissues or cell types measured.

In some embodiments, hypomethylation comprises methylation of less than 1, 5, 10, 15, 20, 25, 30, 35, 40, 45 or 50% of CpG dinucleotides in the locus. Each possibility represents a separate embodiment of the invention. In some embodiments, hypomethylation comprises methylation of less than 40% of CpG dinucleotides in the locus. In some embodiments, hypomethylation comprises methylation of less than 20% of CpG dinucleotides in the locus. In some embodiments, hypomethylation comprises methylation of less than 10% of CpG dinucleotides in the locus.

In some embodiments, hypermethylation comprises methylation of more than 50, 55, 60, 65, 70, 75, 80, 85, 90, 92, 95, 97, or 99% of CpG dinucleotides in the locus. Each possibility represents a separate embodiment of the invention. In some embodiments, hypermethylation comprises methylation of more than 80% of CpG dinucleotides in the locus. In some embodiments, hypermethylation comprises methylation of more than 90% of CpG dinucleotides in the locus. In some embodiments, hypermethylation comprises methylation of more than 95% of CpG dinucleotides in the locus.

In some embodiments, the tissue or cell type that is not leukocytes is selected from: colon, intestine, neutrophils, monocytes, eosinophils, B cells, T cells, CD8 positive cells, and Tregs. In some embodiments, the other tissues or cell types are selected from: colon, intestine, neutrophils, monocytes, eosinophils, B cells, T cells, CD8 positive cells, and Tregs. In some embodiments, the tissues or cell types are selected from: leukocytes, colon, intestine, neutrophils, monocytes, eosinophils, B cells, T cells, CD8 positive cells, and Tregs and the locus comprises differential methylation for one of those tissues or cell types as compared to all of the other tissues or cell types. In some embodiments, the tissues or cell types are selected from: colon, intestine, neutrophils, monocytes, eosinophils, B cells, T cells, CD8 positive cells, and Tregs and the locus comprises differential methylation for one of those tissues or cell types as compared to all of the other tissues or cell types.

In some embodiments, the DNA is identified by its methylation pattern. In some embodiments, the DNA is identified by a methylation specific assay. In some embodiments, the methylation pattern is identified by a methylation specific assay. In some embodiments, the DNA is bisulfite converted. In some embodiments, the methylation pattern is identified after bisulfite conversion. In some embodiments, the bisulfite converted DNA is sequenced.

In some embodiments, the method further comprises sequencing the bisulfite converted DNA. In some embodiments, the DNA is amplified before bisulfite conversion. In some embodiments, the DNA is amplified after bisulfite conversion. In some embodiments, the amplifying is done with a nucleic acid molecule that hybridized to the bisulfite converted DNA and not the unconverted DNA. In some embodiments, the nucleic acid molecule is a primer.

In some embodiments, mammalian DNA is identified by the presence of mammalian DNA sequence. In some embodiments, mammalian DNA is not identified by DNA methylation. In some embodiments, the mammalian specific DNA is primate or human specific. In some embodiments, the primate or human specific DNA sequence is an Alu repeat.

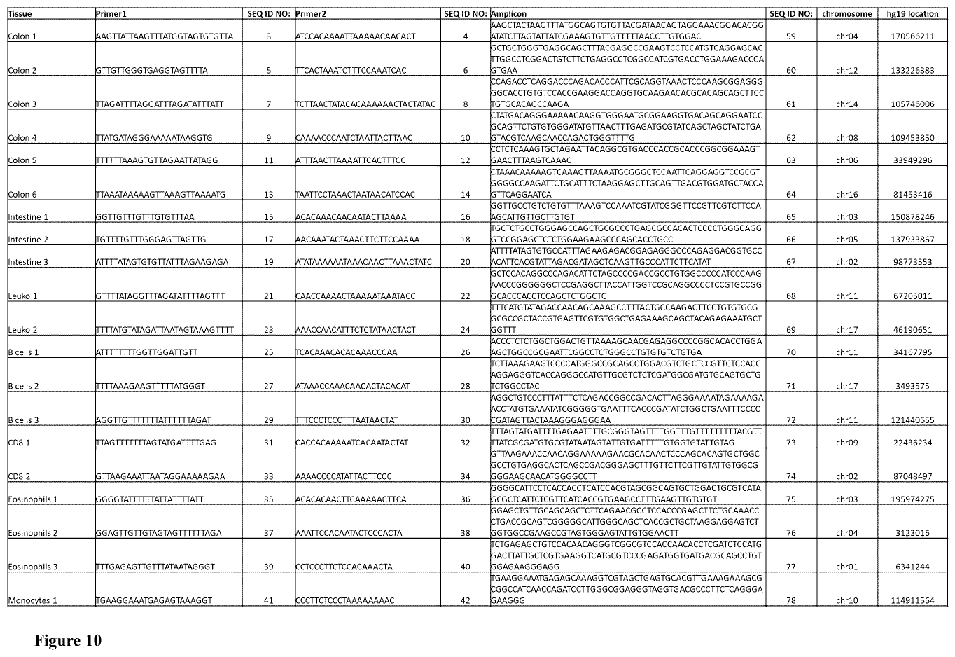

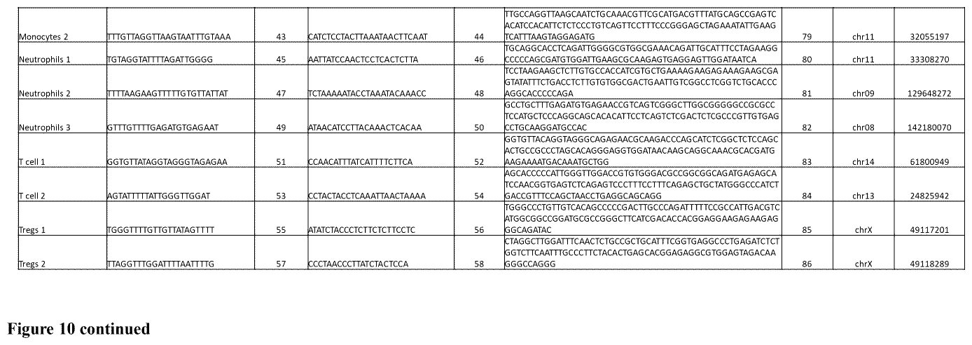

In some embodiments, the locus is selected from the loci provided in FIG. 10. In some embodiments, the locus informative for a given tissue or cell types is provided in FIG. 10. In some embodiments, the locus informative for a given tissue or cell types is provided in International Patent Application WO2019159184 the contents of which are hereby incorporated herein by reference in their entirety. Loci baring tissue specific methylation patterns are well known in the art and any such loci may be used for identifying cfDNA as part of a method of the invention. In some embodiments, a locus is selected from those provided by SEQ ID NO: 59-86.

In some embodiments, a level of DNA above a predetermined threshold indicates the subject suffers from the disease. In some embodiments, a measured level above a predetermined threshold indicates the subject suffers from the disease. In some embodiments, a received level above a predetermined threshold indicates the subject suffers from the disease. In some embodiments, a calculated percentage above a predetermined threshold indicates the subject suffers from the disease. In some embodiments, a percentage above a predetermined threshold indicates the subject suffers from the disease. In some embodiments, a level of DNA below a predetermined threshold indicates the subject does not suffer from the disease. In some embodiments, a measured level below a predetermined threshold indicates the subject does not suffer from the disease. In some embodiments, a received level below a predetermined threshold indicates the subject does not suffer from the disease. In some embodiments, a calculated percentage below a predetermined threshold indicates the subject does not suffer from the disease. In some embodiments, a percentage below a predetermined threshold indicates the subject does not suffer from the disease. In some embodiments, a level of DNA below a predetermined threshold indicates the subject does not suffer from active disease. In some embodiments, a measured level below a predetermined threshold indicates the subject does not suffer from active disease. In some embodiments, a received level below a predetermined threshold indicates the subject does not suffer from active disease. In some embodiments, a calculated percentage below a predetermined threshold indicates the subject does not suffer from active disease. In some embodiments, a percentage below a predetermined threshold indicates the subject does not suffer from active disease.

In some embodiments, the marker is intestine or colon DNA and a level of DNA below a predetermined threshold indicates the subject suffers from the disease. In some embodiments, the marker is intestine or colon DNA and a measured level below a predetermined threshold indicates the subject suffers from the disease. In some embodiments, the marker is intestine or colon DNA and a received level below a predetermined threshold indicates the subject suffers from the disease. In some embodiments, the marker is intestine or colon DNA and a calculated percentage below a predetermined threshold indicates the subject suffers from the disease. In some embodiments, the marker is intestine or colon DNA and a percentage below a predetermined threshold indicates the subject suffers from the disease. In some embodiments, the marker is intestine or colon DNA and a level of DNA above a predetermined threshold indicates the subject does not suffer from the disease. In some embodiments, the marker is intestine or colon DNA and a measured level above a predetermined threshold indicates the subject does not suffer from the disease. In some embodiments, the marker is intestine or colon DNA and a received level above a predetermined threshold indicates the subject does not suffer from the disease. In some embodiments, the marker is intestine or colon DNA and a calculated percentage above a predetermined threshold indicates the subject does not suffer from the disease. In some embodiments, the marker is intestine or colon DNA and a percentage above a predetermined threshold indicates the subject does not suffer from the disease. In some embodiments, the marker is intestine or colon DNA and a level of DNA above a predetermined threshold indicates the subject does not suffer from active disease. In some embodiments, the marker is intestine or colon DNA and a measured level above a predetermined threshold indicates the subject does not suffer from active disease. In some embodiments, the marker is intestine or colon DNA and a received level above a predetermined threshold indicates the subject does not suffer from active disease. In some embodiments, the marker is intestine or colon DNA and a calculated percentage above a predetermined threshold indicates the subject does not suffer from active disease. In some embodiments, the marker is intestine or colon DNA and a percentage above a predetermined threshold indicates the subject does not suffer from active disease.

In some embodiments, the predetermined threshold is a level of DNA in a control stool sample. In some embodiments, the predetermined threshold is an average level of DNA in control stool samples. In some embodiments, the predetermined threshold is a median level of DNA in control stool samples. In some embodiments, a control is a healthy subject. In some embodiments, a control is a subject that does not suffer from the disease. In some embodiments, the control is a healthy child. In some embodiments, the level in the control is of the same DNA as measured or received. It will be understood by a skilled artisan that if a particular cell type's DNA is being examined then the control will be the DNA of that same cell type in the control stool sample. In some embodiments, the control stool sample is a sample from a subject with active disease. In some embodiments, the median mammalian DNA percentage in healthy controls is about 0.5%. In some embodiments, the threshold for mammalian DNA is about 0.5%. In some embodiments, the percent is percent of all DNA in the sample. In some embodiments, the average mammalian DNA percentage in healthy controls is about 1.4%. In some embodiments, the threshold for mammalian DNA is about 1.4%. In some embodiments, the threshold is about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10, 15, 20, 25, 30, 35, 40, 45, 50, 55, 60, 65, 70, 75, 80, 85 or 90%. Each possibility represents a separate embodiment of the invention.

In some embodiments, diagnosing disease comprises classifying the severity of the disease in the subject. In some embodiments, diagnosing UC comprises classifying the severity of the UC in the subject. In some embodiments, total mammalian DNA above a predetermined threshold indicates the subject suffers from moderate to severe UC. In some embodiments, a percentage of mammalian DNA out of total DNA above a predetermined threshold indicates the subject suffers from moderate to severe UC. In some embodiments, a percentage of leukocyte DNA out of total mammalian DNA above a predetermined threshold indicates the subject suffers from moderate to severe UC. In some embodiments, a percentage of neutrophil DNA out of total mammalian DNA above a predetermined threshold indicates the subject suffers from moderate to severe UC. In some embodiments, total intestine DNA above a predetermined threshold indicates the subject suffers from moderate to severe UC. In some embodiments, a percentage of intestine DNA out of total mammalian DNA below a predetermined threshold indicates the subject suffers from moderate to severe UC. In some embodiments, total colon DNA above a predetermined threshold indicates the subject suffers from moderate to severe UC. In some embodiments, a percentage of colon DNA out of total mammalian DNA below a predetermined threshold indicates the subject suffers from moderate to severe UC. In some embodiments, total mammalian DNA at or below a predetermined threshold indicates the subject suffers from mild UC. In some embodiments, a percentage of mammalian DNA out of total DNA at or below a predetermined threshold indicates the subject suffers from mild UC. In some embodiments, a percentage of leukocyte DNA out of total mammalian DNA at or below a predetermined threshold indicates the subject suffers from mild UC. In some embodiments, a percentage of neutrophil DNA out of total mammalian DNA at or below a predetermined threshold indicates the subject suffers from mild UC. In some embodiments, total intestine DNA at or below a predetermined threshold indicates the subject suffers from mild UC. In some embodiments, a percentage of intestine DNA out of total mammalian DNA at or above a predetermined threshold indicates the subject suffers from mild UC. In some embodiments, total colon DNA at or below a predetermined threshold indicates the subject suffers from mild UC. In some embodiments, a percentage of colon DNA out of total mammalian DNA at or above a predetermined threshold indicates the subject suffers from mild UC. In some embodiments, the threshold is a DNA level in a stool sample from a subject with mild UC. In some embodiments, the threshold is a DNA percentage in a stool sample from a subject with mild UC. In some embodiments, the threshold is an average DNA level in a stool sample from subjects with mild UC. In some embodiments, the threshold is an average percentage DNA in a stool sample from subjects with mild UC. In some embodiments, the threshold is an median percentage DNA in a stool sample from subjects with mild UC.

In some embodiments, disease severity is as determined by a noninvasive index. In some embodiments, the noninvasive index is Pediatric Crohn's Disease Activity Index (wPCDAI). In some embodiments, the noninvasive index is Mucosal Inflammation-Non-Invasive (MINI). In some embodiments, the noninvasive index is CD activity index (CDAI).

In some embodiments, the method further comprises treating a subject diagnosed with IBD. In some embodiments, the method further comprises treating a subject diagnosed with UC. In some embodiments, the method further comprises treating a subject diagnosed with CD. In some embodiments, the method further comprises treating a subject not in remission for IBD. In some embodiments, the method further comprises treating a subject not in remission for UC. In some embodiments, the method further comprises treating a subject not in remission for CD.

In some embodiments, treating comprises administering at least one therapeutic agent. In some embodiments, the therapeutic agent is an anti-inflammatory agent. In some embodiments, an anti-inflammatory agent is a steroid. In some embodiments, the steroid is a corticosteroid. In some embodiments, the steroid is prednisone. In some embodiments, the therapeutic agent is an anti-metabolite agent. In some embodiments, the anti-inflammatory agent is an anti-metabolite agent. In some embodiments, the treating comprises dietary intervention. In some embodiments, an anti-inflammatory agent comprises dietary intervention.

In some embodiments, the method further comprises performing a colonoscopy. In some embodiments, the colonoscopy is performed on a subject diagnosed with IBD. In some embodiments, the colonoscopy is performed on a subject diagnosed with UC. In some embodiments, the colonoscopy is performed on a subject diagnosed with CD. In some embodiments, the colonoscopy is performed on a subject diagnosed with remission. In some embodiments, the colonoscopy is performed on a subject diagnosed with active disease. In some embodiments, the colonoscopy is performed on a subject diagnosed not to be in remission. In some embodiments, the colonoscopy is after initial diagnosis. In some embodiments, the colonoscopy is at follow-up. In some embodiments, the diagnosing is a initial diagnosis. In some embodiments, the diagnosis is at follow up.

In some embodiments, the method further comprises not treating a subject not diagnosed with a disease. In some embodiments, the method further comprises discontinuing treatment of a subject diagnosed to be in remission. In some embodiments, the method further comprises not performing a colonoscopy on a subject diagnosed to be in remission. In some embodiments, the method further comprises not performing a colonoscopy on a subject diagnosed to not be in remission.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life.

By another aspect, there is provided a kit comprising reagents for performing a method of the invention.

By another aspect, there is provided a kit comprising:
a. at least one reagent that detects mammalian DNA; and
b. at least one reagent that detects cell type or tissue-specific DNA methylation.

In some embodiments, the kit is for use in a method of the invention. In some embodiments, the kit is configured for use in a method of the invention. In some embodiments, the kit comprises at least 2 reagents. In some embodiments, the kit comprises a plurality of reagents.

In some embodiments, a reagent that detects mammalian DNA is a reagent configured for detecting mammalian DNA. In some embodiments, a reagent that detects mammalian DNA is mammalian DNA detecting reagent. In some embodiments, the reagent is a mammalian DNA specific reagent. In some embodiments, the reagent is specific to mammalian DNA. In some embodiments, the reagent does not detect bacterial DNA. In some embodiments, specific is to the exclusion of bacterial DNA. In some embodiments, mammalian DNA is primate DNA. In some embodiments, primate DNA is human DNA.

In some embodiments, the reagent is sequence specific. In some embodiments, the sequence is a human sequence. In some embodiments, the sequence has less than 50% homology to any sequence in bacteria. In some embodiments, the sequence has less than 50% homology to any sequence in rodents. In some embodiments, the sequence is unique to primates. In some embodiments, primates are humans. In some embodiments, the reagent is specific to an Alu repeat. In some embodiments, an Alu repeat is an Alu repeat region. In some embodiments, the reagent is a nucleic acid molecule. In some embodiments, the reagent is a probe. In some embodiments, the reagent is a primer. In some embodiments, the reagent is a binding agent. In some embodiments, the reagent is complementary to the sequence. In some embodiments, complementary is reverse complementary.

In some embodiments, the cell type or tissue-specific DNA methylation is leukocyte-specific DNA methylation. In some embodiments, the cell type or tissue-specific DNA methylation is neutrophil-specific DNA methylation. In some embodiments, the cell type or tissue-specific DNA methylation is eosinophil-specific DNA methylation. In some embodiments, the cell type or tissue-specific DNA methylation is B cell-specific DNA methylation. In some embodiments, the cell type or tissue-specific DNA methylation is colon-specific DNA methylation. In some embodiments, the cell type or tissue-specific DNA methylation is intestine-specific DNA methylation and monocyte-specific DNA methylation.

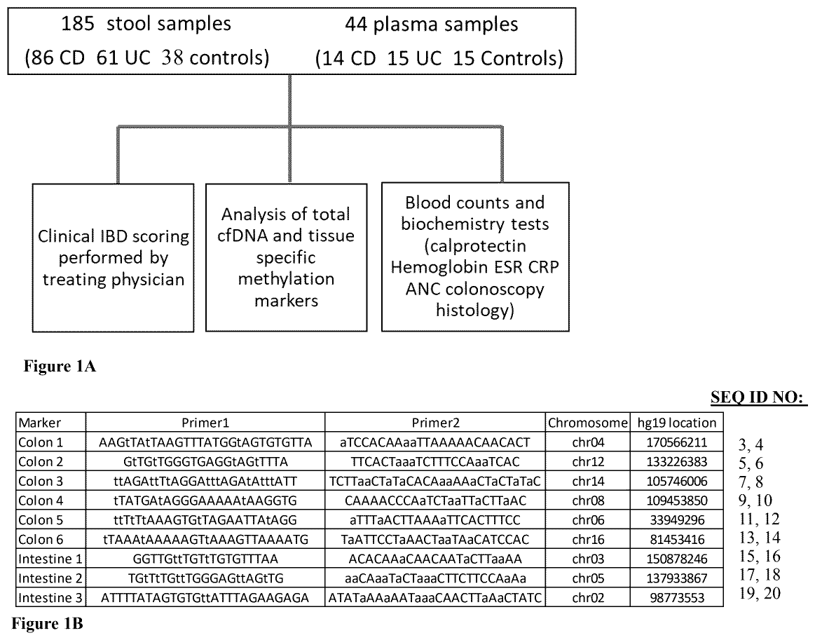

In some embodiments, the reagent is a primer pair. In some embodiments, the primer pair amplifies a locus. In some embodiments, the locus is an informative locus. In some embodiments, the locus is differentially methylated in a cell type or tissue. In some embodiments, differentially methylated is uniquely methylated. In some embodiments, differentially methylated comprises differentially unmethylated. In some embodiments, the primer pair is selected from the sequences provided in SEQ ID NO: 3-58. In some embodiments, the primer pair is selected from the sequences provided in SEQ ID NO: 3-20. In some embodiments, the primer pair is selected from the sequences provided in FIG. 1B. In some embodiments, the primer pair is selected from the sequences provided in FIG. 10.

In some embodiments, the kit further comprises at least one reagent for extracting cfDNA from a sample. In some embodiments, the sample is a stool sample. In some embodiments, the kit further comprises a calibration curve. In some embodiments, the calibration curve is for use of at least one of the reagents. In some embodiments, the calibration curve is an amplification curve. In some embodiments, the kit further comprises instructions for use. In some embodiments, the instructions are for performing a method of the invention.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

### Population:

Children were enrolled at the Pediatric Gastroenterology Institute at Shaare Zedek Medical Center, Jerusalem, Israel. All individuals, male and female up to the age of 18 years old, undergoing endoscopy for suspected IBD or other clinical reasons were offered to enroll in the study. In addition, patients with known Crohn's disease (CD), ulcerative colitis (UC) or IBD unclassified (IBD-U), diagnosed using accepted criteria, who had a clinical evaluation but who had not necessarily undergone colonoscopy at that specific time point were eligible for enrollment. The control group consisted of children who underwent normal colonoscopy as well as otherwise healthy children who were enrolled when visiting the orthopedic clinic. With each sample the following data was recorded in RedCap software: complete blood counts, bicalprotectin, hemoglobin, erythrocyte sedimentation rate (ESR), C-reactive protein (CRP), macroscopic endoscopy (colonoscopy and gastroscopy), histology findings and clinical IBD scoring. Informed consent was given by the child's legal guardian, and patients older than 16 years old were provided with a detailed assent form. The experimental protocol was approved by the SZMC Institutional Review Board and is in agreement with the declaration of Helsinki (SZMC-21-0165).

185 stool samples were collected from 86 children with Crohn's disease (CD), 61 children with ulcerative colitis (UC) and 38 controls individuals. In addition, 44 plasma samples were collected from 14 children with CD, 15 children with UC, and 14 controls. Study design and workflow are shown in FIG. 1A.

### Selection of Cell Type-Specific Methylation Markers:

Cell type-specific methylation biomarkers were selected using comparative analysis of a previously described human cell-type methylome atlas (Loyfer N, et al. “A DNA methylation atlas of normal human cell types.” Nature. 2023 Jan. 4; 613(7943):355-64, the contents of which are hereby incorporated herein by reference in their entirety) to identify loci having more than five CpG sites within 150 bp (given the typical size of cfDNA fragments), with an average methylation value of less than 0.4 in the specific cell type targeted. Based on this analysis, primers were designed to amplify ~100 bp fragments surrounding the informative CpGs. Marker specificity and sensitivity was determined using genomic DNA samples from multiple tissues and cell types, assembled from surgical material or purchased. A cocktail of methylation markers specific for inflammatory and immune cells was described before in Fox-Fisher I, et al. “Remote immune processes revealed by immune-derived circulating cell-free DNA.” eLife. 2021 Nov. 29; 10, the contents of which is hereby incorporated herein by reference in its entirety.

### Sample Collection and Processing:

Stool samples were collected in a test tube without ethanol and transferred within 24 hours at room temperature to storage at −80° C. until DNA extraction. DNA was extracted from 250 mg of feces using the Qiagen Dneasy powerLyzer power Soil Kit. DNA concentration was measured using Qubit® dsDNA HS (High Sensitivity) Assay Kit Qubit Fluorometers (Life Technologies) (Invitrogen), in ng/μl. To quantify DNA, the concentration obtained for 1 μl was multiplied by 70 (elution volume). Finally, DNA content in 1 gram of feces was calculated. For example, a concentration of 100 ng/μl in a sample extracted from 250 mg of feces is transformed as follows: 100 ng/μl*70 μl=7000 ng in 250 mg. Therefore, 28 micrograms DNA in 1 gr stool.

Blood samples were collected in EDTA tubes, centrifuged twice and plasma was stored at-20c as described. cfDNA was extracted from plasma using the Qiasymphony liquid handling robot and measured using Qubit as described above.

### Quantification of Human DNA:

EvaGreen ddPCR, was used to quantify the number of human DNA molecules according to manufacturer instructions (BIORAD Droplet Digital™ PCR Technology).

Primers were designed for the human SFPTC-1 gene. ddPCR was run on 5 ng total stool DNA and included a negative control (no template control, NTC) and a positive control (PTC), with each cutoff threshold set manually for each sample using acceptance criteria defined during the optimization of each experiment, using QuantaSoft™ software (version 1.7.4). The concentration of human DNA per gram stool was infered assuming that each copy of human SFPTC-1 identified in stool represented one genome equivalent, or 3.3 picograms of human DNA. The primers for the ddPCR reaction were: SFTPC1 (forward): 5′-AGCAAAGAGGTCCTGATGGAGA-3′ (SEQ ID NO: 1) and (reverse) 5′-GCAGGGCCCATCACACACAT-3′ (SEQ ID NO: 2).

### Bisulfite Treatment and Multiplex PCR:

DNA was treated with bisulfite using EZ DNA Methylation-Gold™ (Zymo Research), according to the manufacturer's instructions, and eluted in 20 μl elution buffer.

To efficiently amplify and sequence multiple targets from bisulfite-treated cfDNA, a two-step multiplex PCR protocol as previously described in Neiman D, et al. “Multiplexing DNA methylation markers to detect circulating cell-free DNA derived from human pancreatic β cells.” JCI Insight. 2020 Jul. 23; 5(14), herein incorporated by reference in its entirety, was applied. Briefly, up to 30 primer pairs (each containing a short adapter) were used in one PCR reaction to amplify regions of interest from bisulfite-treated DNA, independent of methylation status. Primers were 18-30 base pairs (bp) with primer melting temperatures ranging from 58-62° C. (see FIG. 1B for sequences of SEQ ID NO: 3-20). To maximize amplification efficiency and minimize primer interference, the primers were designed with 25 bp adaptors comprising Illumina TruSeq Universal Adaptors without index tags. All primers were mixed in the same reaction tube. For each sample, the PCR reaction was prepared using the QIAGEN Multiplex PCR Kit according to manufacturer instructions with 10 μl of bisulfite treated cfDNA. Reaction conditions for the first round of PCR were: 95° C. for 15 minutes, followed by 30 cycles of 95° C. for 30 seconds, 57° C. for 3 minutes and 72° C. for 1.5 minutes, followed by 10 minutes at 68° C.

This was followed by an exonuclease step and a reaction using primers specific to the adapters, which added barcodes and Illumina Universal Adaptors. The products of the first PCR reaction were treated with Exonuclease I (ThermoScientific) for primer removal according to manufacturer instructions. Purified PCR products were amplified using one unique TruSeq Universal Adaptor primer pair per sample to add a unique index barcode to enable sample pooling for multiplex Illumina sequencing. The PCR reaction was prepared using 2×PCRBIO HS Taq Mix Red Kit (PCR Biosystems) according to manufacturer instructions. Reaction conditions for the second round of PCR were: 95° C. for 2 minutes, followed by 15 cycles of 95° C. for 30 seconds, 59° C. for 1.5 minutes, 72° C. for 30 seconds, followed by 10 minutes at 72° C. The PCR products were then pooled, run on 3% agarose gels with ethidium bromide staining, and extracted by a Zymo gel Recovery kit. Primers for immune and inflammatory cell types were previously described in Fox-Fisher et al. Primers for intestinal markers are provided in FIG. 1B.

### Next Generation Sequencing:

Pooled PCR products were subjected to next-generation sequencing (NGS) using the MiSeq Reagent Kit v2 (Illumina) or the NextSeq 500/550 v2 Reagent Kit (Illumina). Sequenced reads were separated by barcode, and aligned to the target sequence with Bismark, using an available computational pipeline (github.com/Joshmoss11/btseq) Reads were quality filtered based on Illumina quality scores and identified as having at least 80% similarity to the target sequences and containing all the expected CpGs. Samples with less than 1000 total reads were filtered-out. CpGs were considered methylated if “CG” was read and unmethylated if “TG” was read. Proper bisulfite conversion was assessed by analyzing methylation of non-CpG cytosines. The fraction of molecules in which all CpG sites were unmethylated was then determined. The fraction obtained was multiplied by the concentration of cfDNA measured in each sample, to obtain the concentration of tissue-specific cfDNA from each donor.

### Example 1: No Evidence of Altered Plasma cfDNA in IBD

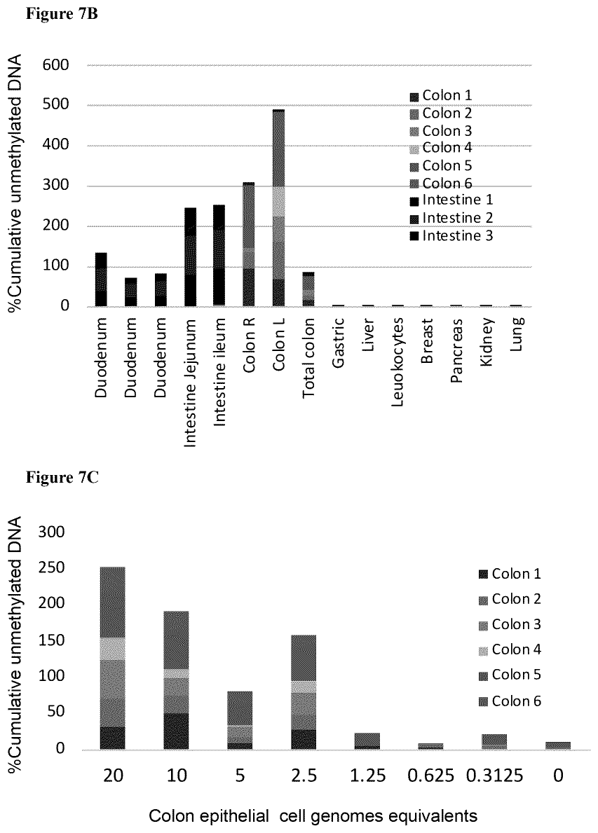

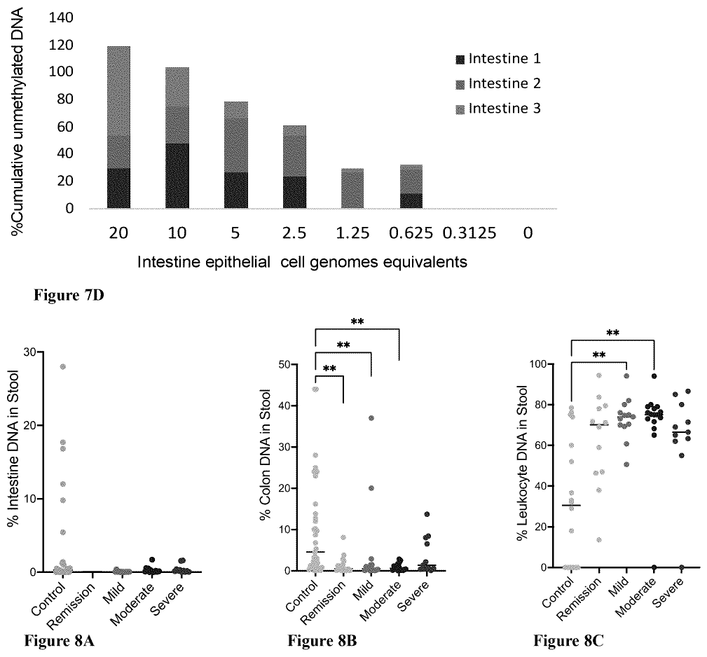

Based on the human cell type methylome atlas previously described, genomic loci were selected, each containing 3-5 CpGs, whose DNA methylation patterns were unique to colon epithelial cells (n=6 loci, unmethylated only in DNA from the left and right colon) or to small intestine epithelial cells (n=3 loci, uniquely unmethylated in DNA from the duodenum, jejunum and ileum) compared with all other cell types in the atlas (see FIG. 10). All loci were unmethylated in the cell type of interest and methylated in all other tissue, as is typical to cell type-specific methylation markers. Primers were designed to amplify marker loci from bisulfite-treated DNA, and validated specificity using PCR-sequencing on genomic DNA from multiple cell types. As shown in FIGS. 7A-7D, markers selected were indeed specific. Colon markers were unmethylated only in DNA from the left and right colon. Intestinal markers were unmethylated specifically in DNA from the ileum, jejunum and duodenum and methylated in all other cell types including gastric epithelium. Additionally, two novel pan-leukocyte methylation markers were designed that were demethylated specifically in leukocytes and in DNA from bulk tissue, but not in isolated epithelial cells (FIG. 7A).

To assess the sensitivity of the markers, spike-in experiments were conducted. The assay was able to identify colon or small intestine DNA that is spiked into leukocyte DNA, even when comprising as little as 0.08% of the total, and when only 2.5 intestinal genome equivalents were present (FIG. 7C-7D). Finally, conditions were established for multiplex PCR, enabling the co-amplification and sequencing of all 9 intestinal markers from the same bisulfite-treated DNA samples. In the experiments described below, the intestinal marker cocktail was typically used on ⅓ of the DNA from each sample, and the previously described immune/inflammatory marker cocktail, along with two pan-leukocyte markers, was used on another ⅓.

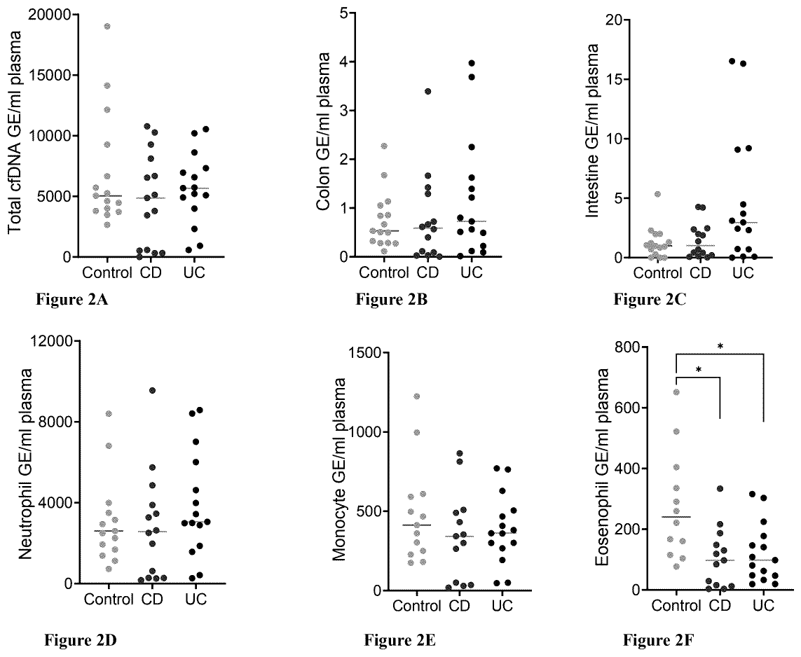

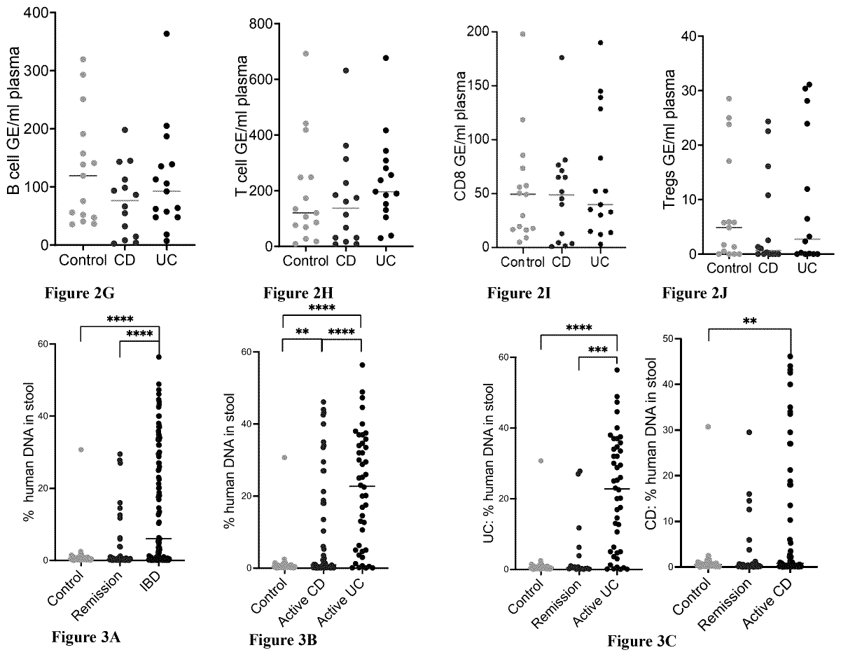

Children with either UC or CD exhibited a total cfDNA concentration that did not significantly differ from controls (healthy children and patients with irritable bowel syndrome with normal colonoscopy and no evidence of inflammation), averaging at 18 ng/ml-consistently higher than healthy adults, as previously shown for children (FIG. 2A). This suggests that intestinal damage and associated local inflammation and immune cell turnover do not lead to a massive excess release of cfDNA to blood. To test this idea at higher resolution, cell type-specific methylation markers were applied.

Human intestine undergoes turnover every 3-5 days by renewal from stem cells and shedding of cells to the lumen of gut. Consistent with this architecture, the plasma methylome deconvolution as well as the new targeted assay for colon and small intestine DNA markers revealed no evidence of intestinal epithelial DNA in the plasma of healthy children (~1 GE/ml, or less than 0.1% of the total) (FIG. 2B-2C). Strikingly, the plasma of children with IBD also lacked intestinal cfDNA (FIG. 2B-2C), except for elevated small intestine cfDNA in some patients with UC, which had no distinct clinical or biochemical features.

Further examination of the concentration of cfDNA from inflammatory and immune cell types including neutrophils, monocytes, B cells, T cells, and CD8 and regulatory T cells was performed. No differences were observed between plasma samples from controls and those from patients with UC or CD, with the exception of an unexplained reduction of eosinophil cfDNA in patients with UC and CD compared with controls (FIG. 2D-2J). These findings suggest that intestinal injury and inflammation in IBD do not leave a substantial mark on plasma cfDNA patterns.

### Example 2: Human DNA is Elevated in the Stool of Children with IBD

Given the lack of distinct cfDNA signals in the plasma of patients with IBD, it was hypothesized that in such patients the intestinal turnover clearance route is preserved, such that material from dying cells—epithelial or inflammatory—is shed in stool.

Stool DNA is dominated by the microbiome. A quantitative PCR assay for human DNA in stool was established, and it was found that the stool of the control children contained 0.5% (median) or 1.4% (average) human DNA, in general agreement with previous reports (FIG. 3A).

Strikingly, patients with active IBD exhibited a higher proportion of human DNA in stool (median 1.23%, average 11.36%) compared with controls, while patients in remission had normal levels of human DNA in stool (FIG. 3A). Upon segregating the IBD cohort into patients with active CD and active UC, a higher than normal proportion of human DNA was observed in stool samples from both groups. The magnitude of the effect was more pronounced in patients with active UC compared to CD (on average 9.7% human DNA in active CD, and 21% in active UC; FIG. 3B-3C).

It was also asked if the presence of human DNA in stool correlated with disease severity. As shown in FIG. 3D, UC patients scored as having moderate or severe disease had higher levels of human DNA in stool compared with controls and patients in remission. Patients with CD showed a similar trend that did not reach statistical significance. In summary, the stool of patients with active UC and CD contains an increased fraction of human DNA, which correlates with disease severity.

### Example 3: Methylation Markers Reveal Tissue Origins of Stool DNA in IBD

Next, the relative contribution of DNA from intestinal epithelial cells and inflammatory/immune cells to stool was assessed. Surprisingly, human DNA in the stool of healthy individuals was dominated by leukocytes, with only a minority derived from colon and small intestine epithelial cells (FIG. 4A). Strikingly, patients with IBD had a further increase in the proportion of human DNA from leukocytes, while the contribution of colon and intestinal was reduced (FIG. 4A). The same observations were made when IBD patients were divided into UC and CD. In both patient groups the proportion of leukocyte DNA was increased while the proportion of DNA from the intestine was reduced (FIG. 4B). The major contributor to elevated leukocyte DNA percentage was neutrophil DNA; DNA from eosinophils was also elevated in the stool of patients with UC and CD, but this did not reach statistical significance, neither did DNA from other inflammatory or immune cell types (FIG. 4B).

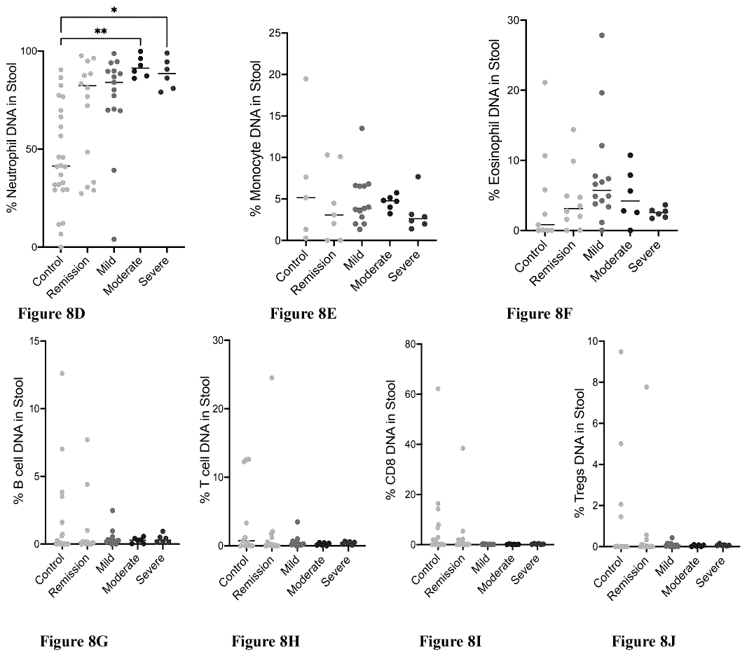

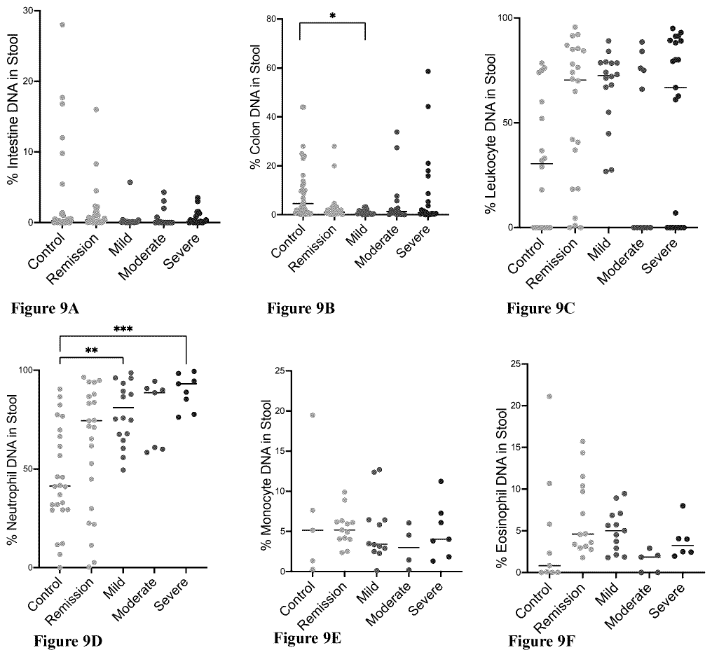

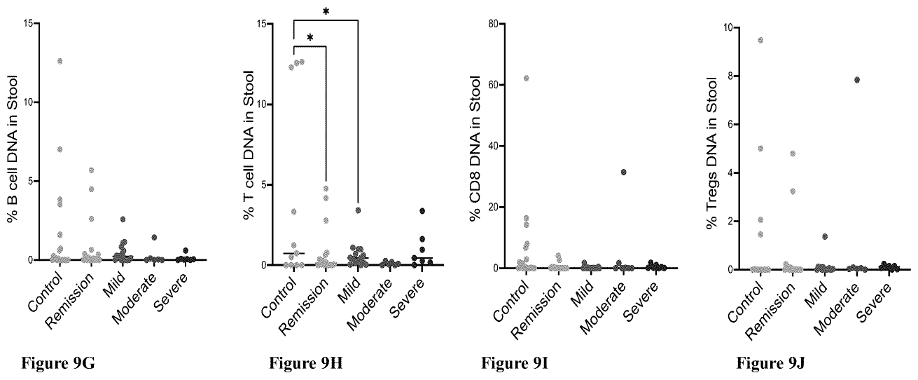

The patterns of methylation in stool as a function of disease severity were also examined. The concentrations of markers for leukocytes, and specifically neutrophils, were higher in UC patients with moderate or severe disease compared with controls, while the concentration of DNA from colon epithelial cells was reduced (FIG. 8A-8J). CD patients also had elevated concentration of neutrophil DNA that was higher in severe disease (FIG. 9A-9J).

### Example 4: Correlations Between Clinical and Stool DNA Measures

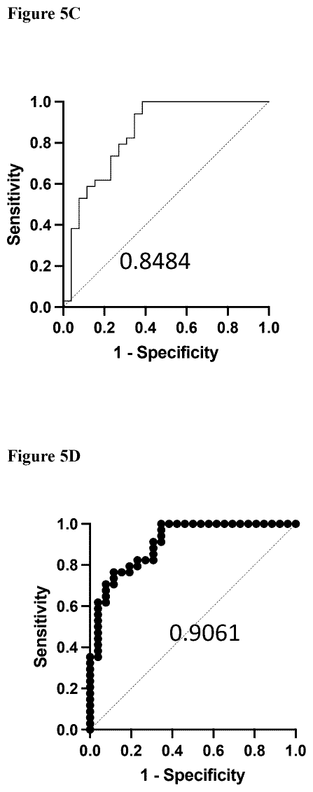

To assess whether human stool DNA parameters reflect clinical features of IBD, a matrix was generated that describes the strength of correlation between each stool DNA parameter and each clinical parameter separately, for both UC and CD (FIG. 5A-5B). DNA parameters were strongly correlated to each other. In UC, DNA parameters correlated strongly with PUCAI and with calprotectin, and more weakly with CRP, albumin and ESR. The correlations between stool DNA measured and clinical parameters were weaker in CD. Thus, the presence of human DNA in stool from specific sources correlates partially with clinical measures of IBD.

The predictive ability of human DNA in stool was evaluated. Human DNA amounts could predict the presence of IBD. FIG. 5C shows a ROC curves demonstrating the percentage of neutrophil DNA in stool is able to distinguish active CD from healthy controls with high sensitivity and specificity (AUC-0.8484). This ability is further improved when logistic regression is used to take into account both the percentage of neutrophil DNA in stool and the percentage of human DNA in stool (AUC=0.906) (FIG. 5D).

### Example 5: DNA Features are More Strongly Elevated in IBD Patients that Underwent Colonoscopy

As the grading of disease severity is based on clinical scores and not on a single gold standard measure, an analysis was performed only in children who underwent colonoscopy that revealed IBD. This allowed the correlation of the levels of human DNA in stool to the actual tissue damage observed by colonoscopy and histology, compared with controls (healthy children and children with IBS). Children in this smaller group (total n=29 children with UC, 39 children with CD) underwent colonoscopy for initial diagnosis, at exacerbation or as routine follow up and were compared to healthy controls and children in remission with normal colonoscopy (n=42).

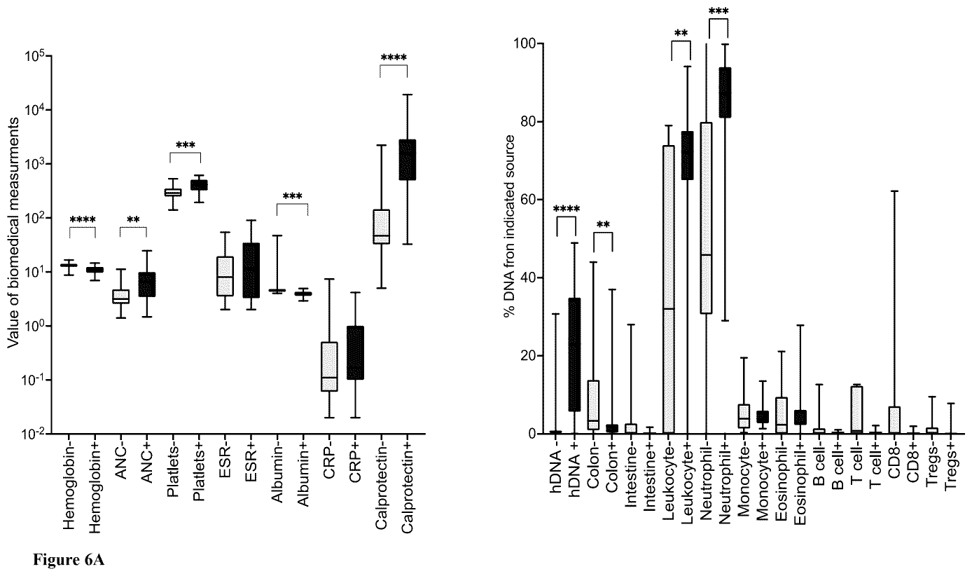

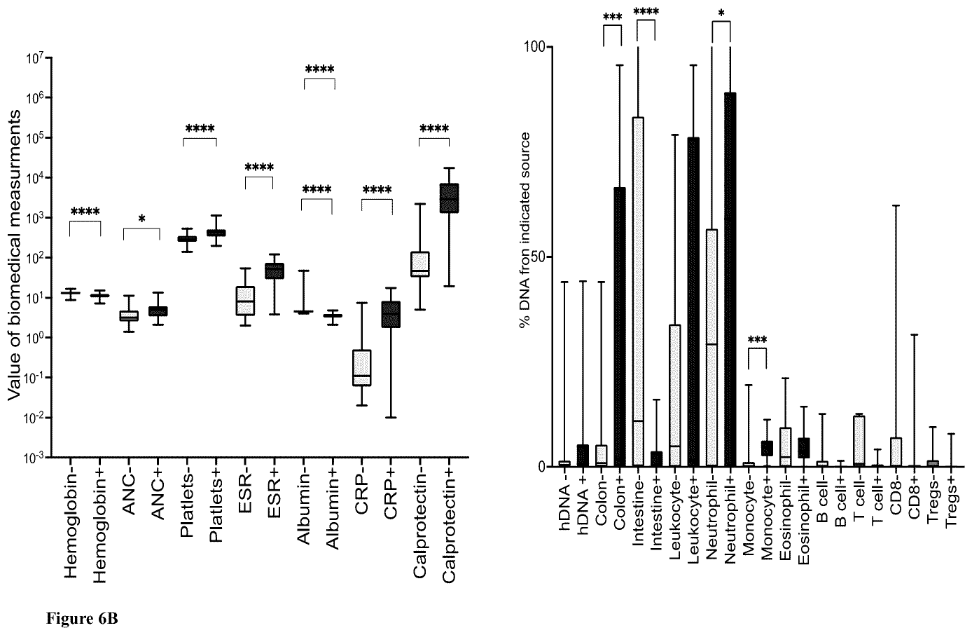

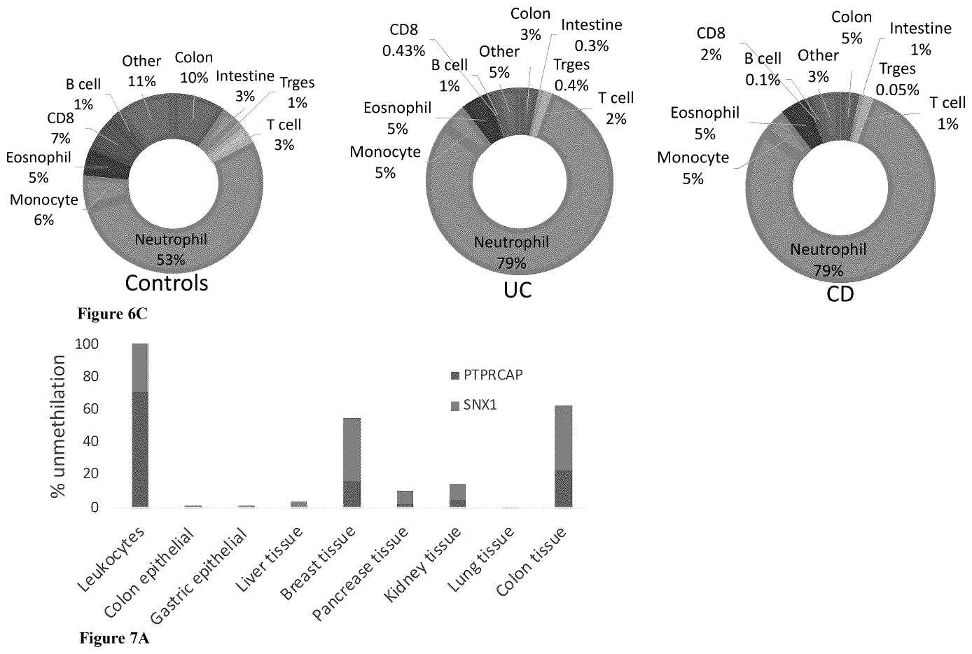

Children with colonoscopy-verified UC displayed a dramatic elevation in the fraction of human DNA in stool, which was driven by an increased fraction of leukocyte DNA in stool, mostly originating in neutrophils. This was accompanied by a reduced fraction of colon DNA (FIGS. 6A and 6C). Children with colonoscopy-verified CD also had an elevated proportion of DNA of human origin in stool (FIGS. 6B and 6C). The proportion of both neutrophil and monocyte DNA was elevated in the stool of CD patients, and the proportion of DNA of colon and small intestine origin was reduced.

Thus, the proportion of human DNA in stool and the relative contribution of inflammatory and epithelial cells to stool DNA distinguish controls from colonoscopy-verified UC and CD, strongly supporting validity of the DNA assay.

### Example 6: Human cfDNA is Also Predictive of IBD in Adults

Stool samples are also collected from adults with IBD, both CD and UC. cfDNA is extracted from the stool and the percentage of total cfDNA that is human is elevated in subjects that suffer from CD and UC as compared to healthy controls. Similarly, the percentage of human cfDNA that is from leukocyte and specifically neutrophils is elevated in IBD subject and the percentage that is from colon and intestine is decreased. Thus, the markers predictive in children are also predictive in adults. The severity of the disease can also be predicted/evaluated in adults as in children.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of diagnosing inflammatory bowel disease (IBD) in a subject in need thereof, the method comprising receiving a stool sample from said subject and measuring DNA levels in said stool sample wherein a proportion of human DNA out of total DNA in the stool sample, or a proportion of leukocyte DNA, neutrophil DNA, eosinophil DNA or monocyte DNA out of total human DNA in the stool sample, or a combination thereof above a predetermined threshold indicates the subject suffers from IBD and treating a subject diagnosed with IBD, with at least one anti-inflammatory agent or performing a colonoscopy on a subject diagnosed with IBD; thereby diagnosing IBD in a subject.

2. The method of claim 1, wherein said IBD is Ulcerative colitis (UC), Crohn's Disease (CD) or both.

3. The method of claim 1, wherein said predetermined threshold is a proportion of DNA in a stool sample from a healthy subject.

4. The method of claim 2, wherein said diagnosing UC or CD further comprises classifying the severity of UC or CD in said subject, wherein a proportion of human DNA, leukocyte DNA, or neutrophil DNA above a predetermined threshold indicates said subject suffers from moderate or severe UC or CD and wherein said predetermined threshold is a proportion of DNA in a stool sample from a subject with mild UC or CD.

5. A method of diagnosing IBD remission in a subject that suffered from IBD, the method comprising receiving a stool sample from said subject and measuring DNA levels in said stool sample wherein a proportion of human DNA out of total DNA in the stool sample, or a proportion of leukocyte DNA, colon DNA, or neutrophil cfDNA out of total human DNA in the stool sample below a predetermined threshold indicates the subject is in IBD remission and discontinuing treatment of a said subject diagnosed to be in remission; thereby diagnosing IBD remission in a subject.

6. The method of claim 5, wherein said IBD is UC, said predetermined threshold is a proportion of DNA in a stool sample from a subject suffering from active disease or both.

7. (canceled)

8. The method of claim 1, wherein said subject is a child.

9. The method of claim 1, further comprising extracting DNA from said stool sample and sequencing said extracted DNA.

10. (canceled)

11. The method of claim 1, wherein DNA from a tissue or cell type that is not leukocytes comprises differential methylation of at least one locus as compared to methylation in leukocytes.

12. The method of claim 11, wherein at least one of:
a. said locus comprises at least two CpG dinucleotides;
b. said locus comprises at least 5 CpG dinucleotides;
c. said locus is hypomethylated in said tissue or cell type and hypermethylated in leukocytes;
d. said locus comprises methylation of less than 40% of CpG dinucleotide in said tissue or cell type and methylation of greater than 90% of CpG dinucleotides in leukocytes;
e. DNA from a tissue or cell type comprises differential methylation of said at least one locus as compared to at least 5 other tissues/cell types; and
f. DNA from a tissue or cell type comprises differential methylation of said at least one locus as compared to at least 5 other tissues/cell types and said locus is hypomethylated in said tissue or cell type and hypermethylated in said at least 5 other tissues/cell types.

13. (canceled)

14. (canceled)

15. (canceled)

16. (canceled)

17. The method of claim 11, wherein DNA from a tissue or cell type comprises differential methylation of said at least one locus as compared to at least 5 other tissues/cell types, optionally wherein said locus is hypomethylated in said tissue or cell type and hypermethylated in said at least 5 other tissues/cell types.

18. (canceled)

19. (canceled)

20. The method of claim 17, wherein said tissue or cell type that is not leukocytes and said at least 5 other tissues/cell types are selected from colon, intestine, neutrophils, monocytes, eosinophils, B cells, T cells, CD8 positive cells, and Tregs.

21. (canceled)

22. The method of claim 11, wherein said DNA is identified by a methylation specific assay or wherein said DNA is bisulfite converted and said method further comprises sequencing said bisulfite converted DNA or amplifying said DNA with a nucleic acid molecule that hybridizes to said bisulfite converted cfDNA and not said unconverted DNA.

23. (canceled)

24. The method of claim 1, wherein said human DNA is identified by the presence of human specific DNA sequence.

25. The method of claim 11, wherein said locus comprises a DNA sequence selected from SEQ ID NO: 59-86.

26. The method of claim 1, wherein a combination of said proportion of human DNA and said proportion of neutrophil DNA above a predetermined threshold indicates the subject suffers from IBD.

27. (canceled)

28. The method of claim 1, wherein said anti-inflammatory agent is selected from: steroids, an anti-metabolite agent, and dietary intervention.

29. (canceled)

30. (canceled)

31. A kit comprising:
a. at least one reagent that detects human DNA and not bacterial DNA; and
b. at least one reagent that detects cell type or tissue-specific DNA methylation; and
optionally at least one of: a reagent for extracting DNA from stool, a calibration curve for use of said reagents or instructions for use.

32. The kit of claim 31, wherein said at least one reagent that detects human DNA and not bacterial DNA is sequence specific to a human DNA sequence, said cell type or tissue-specific DNA methylation is selected from leukocyte-specific DNA methylation, neutrophil-specific DNA methylation, eosinophil-specific DNA methylation, B cell-specific DNA methylation, colon-specific DNA methylation, intestine-specific DNA methylation and monocyte-specific DNA methylation or both.

33. (canceled)

34. The kit of claim 32, wherein said reagent that detect cell type or tissue-specific DNA methylation is a primer pair that amplifies a locus of at least 150 and at most 500 nucleotides that comprises at least two CpG dinucleotides that are uniquely methylated or unmethylated in said cell type or tissue, optionally wherein said primer pair is selected from the sequences provided in SEQ ID NO: 3-58.

35. (canceled)

36. (canceled)

37. (canceled)
